SEQUENCE LISTING (283 sequences)

SEQ ID NO: 1 - DNA, 6911 bp, Homo sapiens
  CDS (374)...(6613)
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 2 - protein, 2080 aa, Homo sapiens
MLRVFILYAENVHTPDTDISDAYCSAVFAGVKKRTKVIKNSVNPVWNEGFEWDLKGIPLDQGSELHVVVKDHETMGRNRFLGEAKVPLREVLATPSLSASFNAPLLDTKKQPTGASLVLQVSYTPLPGAVPLFPPPTPLEPSPTLPDLDVVADTGGEEDTEDQGLTGDEAEPFLDQSGGPGAPTTPRKLPSRPPPHYPGIKRKRSAPTSRKLLSDKPQDFQIRVQVIEGRQLPGVNIKPVVKVTAAGQTKRTRIHKGNSPLFNETLFFNLFDSPGELFDEPIFITVVDSRSLRTDALLGEFRMDVGTIYREPRHAYLRKWLLLSDPDDFSAGARGYLKTSLCVLGPGDEAPLERKDPSEDKEDIESNLLRPTGVALRGAHFCLKVFRAEDLPQMDDAVMDNVKQIFGFESNKKNLVDPFVEVSFAGKMLCSKILEKTANPQWNQNITLPAMFPSMCEKMRIRIIDWDRLTHNDIVATTYLSMSKISAPGGEIEEEPAGAVKPSKASDLDDYLGFLPTFGPCYINLYGSPREFTGFPDPYTELNTGKGEGVAYRGRLLLSLETKLVEHSEQKVEDLPADDILRVEKYLRRRKYSLFAAFYSATMLQDVDDAIQFEVSIGNYGNKFDMTCLPLASTTQYSRAVFDGCHYYYLPWGNVKPVVVLSSYWEDISHRIETQNQLLGIADRLEAGLEQVHLALKAQCSTEDVDSLVAQLTDELIAGCSQPLGDIHETPSATHLDQYLYQLRTHHLSQITEAALALKLGHSELPAALEQAEDWLLRLRALAEEPQNSLPDIVIWMLQGDKRVAYQRVPAHQVLFSRRGANYCGKNCGKLQTIFLKYPMEKVPGARMPVQIRVKLWFGLSVDEKEFNQFAEGKLSVFAETYENETKLALVGNWGTTGLTYPKFSDVTGKIKLPKDSFRPSAGWTWAGDWFVCPEKTLLHDMDAGHLSFVEEVFENQTRLPGGQWIYMSDNYTDVNGEKVLPKDDIECPLGWKWEDEEWSTDLNRAVDEQGWEYSITIPPERKPKHWVPAEKMYYTHRRRRWVRLRRRDLSQMEALKRHRQAEAEGEGWEYASLFGWKFHLEYRKTDAFRRRRWRRRMEPLEKTGPAAVFALEGALGGVMDDKSEDSMSVSTLSFGVNRPTISCIFDYGNRYHLRCYMYQARDLAAMDKDSFSDPYAIVSFLHQSQKTVVVKNTLNPTWDQTLIFYEIEIFGEPATVAEQPPSIVVELYDHDTYGADEFMGRCICQPSLERMPRLAWFPLTRGSQPSGELLASFELIQREKPAIHHIPGFEVQETSRILDESEDTDLPYPPPQREANIYMVPQNIKPALQRTAIEILAWGLRNMKSYQLANISSPSLVVECGGQTVQSCVIRNLRKNPNFDICTLFMEVMLPREELYCPPITVKVIDNRQFGRRPVVGQCTIRSLESFLCDPYSAESPSPQGGPDDVSLLSPGEDVLIDIDDKEPLIPIQEEEFIDWWSKFFASIGEREKCGSYLEKDFDTLKVYDTQLENVEAFEGLSDFCNTFKLYRGKTQEETEDPSVIGEFKGLFKIYPLPEDPAIPMPPRQFHQLAAQGPQECLVRIYIVRAFGLQPKDPNGKCDPYIKISIGKKSVSDQDNYIPCTLEPVFGKMFELTCTLPLEKDLKITLYDYDLLSKDEKIGETVVDLENRLLSKFGARCGLPQTYCVSGPNQWRDQLRPSQLLHLFCQQHRVKAPVYRTDRVMFQDKEYSIEEIEAGRIPNPHLGPVEERLALHVLQQQGLVPEHVESRPLYSPLQPDIEQGKLQMWVDLFPKALGRPGPPFNITPRRARRFFLRCIIWNTRDVILDDLSLTGEKMSDIYVKGWMIGFEEHKQKTDVHYRSLGGEGNFNWRFIFPFDYLPAEQVCTIAKKDAFWRLDKTESKIPARVVFQIWDNDKFSFDDFLGSLQLDLNRMPKPAKTAKKCSLDQLDDAFHPEWFVSLFEQKTVKGWWPCVAEEGEKKILAGKLEMTLEIVAESEHEERPAGQGRDEPNMNPKLEDPRRPDTSFLWFTSPYKTMKFILWRRFRWAIILFIILFILLLFLAIFIYAFPNYAAMKLVKPFS

SEQ ID NO: 3 - DNA, 5915 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttg

SEQ ID NO: 4 - DNA, 20 bp, Homo sapiens
tgggacctcaaagggcatcc

SEQ ID NO: 5 - DNA, 20 bp, Homo sapiens
accatgctgtaggatgtgga

SEQ ID NO: 6 - DNA, 20 bp, Homo sapiens
gggaggtgaagcaacttcaa

SEQ ID NO: 7 - DNA, 20 bp, Homo sapiens
ctcacggggtagaagatgag

SEQ ID NO: 8 - DNA, 20 bp, Homo sapiens
cagggccgagatgagcccaa

SEQ ID NO: 9 - DNA, 20 bp, Homo sapiens
acatcaagggtcctggatga

SEQ ID NO: 10 - DNA, 20 bp, Homo sapiens
ctgtggcggtgtttccggtg

SEQ ID NO: 11 - DNA, 20 bp, Homo sapiens
acagacgtgcgttatcgttc

SEQ ID NO: 12 - DNA, 20 bp, Homo sapiens
aagactgagcaaaatcccag

SEQ ID NO: 13 - DNA, 6912 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgtaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 14 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgtaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 15 - DNA, 6910 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaagcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: SEQ ID O 16 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacggggtagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 17 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgagatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 18 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaagggtcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 19 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggtgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 20 - DNA, 6911 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcgttatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 21 - DNA, 6909 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 22 - DNA, 20 bp, Homo sapiens
tgggacctcaagggcatccc

SEQ ID NO: 23 - DNA, 20 bp, Homo sapiens
accatgctgcaggatgtgga

SEQ ID NO: 24 - DNA, 20 bp, Homo sapiens
gggaggtgaaggcaacttca

SEQ ID NO: 25 - DNA, 20 bp, Homo sapiens
ctcacgggggagaagatgag

SEQ ID NO: 26 - DNA, 20 bp, Homo sapiens
ctgtggcggcgtttccggtg

SEQ ID NO: 27 - DNA, 20 bp, Homo sapiens
acatcaaggatcctggatga

SEQ ID NO: 28 - DNA, 20 bp, Homo sapiens
ctgtggcggcgtttccggtg

SEQ ID NO: 29 - DNA, 20 bp, Homo sapiens
acagacgtgcattatcgttc

SEQ ID NO: 30 - DNA, 20 bp, Homo sapiens
aagactgagagcaaaatccc

SEQ ID NO: 31 - DNA, 507 bp, Homo sapiens
tcgaccgcccagccaggtgcaaaatgccgtgtcattgggagactccgcagccggagcattagattacagctcgacggagctcgggaagggcggcgggggtggaagatgagcagaagcccctgttctcggaacgccggctgacaagcggggtgagcgcaggcggggcggggacccagcctagcccactggagcagccgggggtggcccgttcccctttaagagcaactgctctaagccaggagccagagattcgagccggcctcgcccagccagccctctccagcgaggggacccacaagcggcgcctcggccctcccgacctttccgagccctctttgcgccctgggcgcacggggccctacacgcgccaagcatgctgagggtcttcatcctctatgccgagaacgtccacacacccgacaccgacatcagcgatgcctactgctccgcggtgtttgcaggtaggaggggccgaccaccctcgccggggtcggggtggggtagagg

SEQ ID NO: 32 - DNA, 183 bp, Homo sapiens
aaaggcgggatgtgtctctccattctcccttttgtgtctcttgtaggggtgaagaagagaaccaaagtcatcaagaacagcgtgaaccctgtatggaatgaggtatgtgagtttttctccttccttttctctctgtctgctgcagggggcttgggaggaggtgccttctcagcagtgtccttg

SEQ ID NO: 33 - DNA, 264 bp, Homo sapiens
cattcatgaatgcctactcagtgccctggtggcacgaaggtgaaccagacacagtctcttctcctagagggccataggttaagatgccttttctctttttcttccagggatttgaatgggacctcaagggcatccccctggaccagggctctgagcttcatgtggtggtcaaagaccatgagacgatggggaggaacaggtaaggtggccagaggggggtgctccatggcttgaaggtgcaggtaggattgtggagtatacaga

SEQ ID NO: 34 - DNA, 223 bp, Homo sapiens
cagaagagccagggtgccttaggctagttttctacatttgacttctctctcctctcaggttcctgggggaagccaaggtcccactccgagaggtcctcgccacccctagtctgtccgccagcttcaatgcccccctgctggacaccaagaagcagcccacaggggtaagtgcccatcagcctctgccaggttaaggtccaaggcattgccaggtggcttcctc

SEQ ID NO: 35 - DNA, 224 bp, Homo sapiens
cagtggtccgaggccagcgcaccaacctgtcccccacgtctcatctcttccaggcctcgctggtcctgcaggtgtcctacacaccgctgcctggagctgtgcccctgttcccgccccctactcctctggagccctccccgactctgcctgacctggatgtagtggcaggtgggtagcccacgttggcctggctgggccccagcaagaatggccggcagtggcac

SEQ ID NO: 36 - DNA, 315 bp, Homo sapiens
aggggcaggggcagggccagagggccaggcctcattagggccctctcctcttagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccaggtgatgaacgggctttctctgaccccaggctcctcttcagccatcagctgcgggt

SEQ ID NO: 37 - DNA, 249 bp, Homo sapiens
ccagtggtgagatggtccctgagatttctgactcttggggtggatggtgggtggtccttaactcttcccccttctggctttcagatcagggtccaggtgatcgaggggcgccagctgccgggggtgaacatcaagcctgtggtcaaggttaccgctgcagggcagaccaagcggacgcggatccacaagggaaacagcccactcttcaatgaggtgggagacatggggcatgagggcagaaccttgtgg

SEQ ID NO: 38 - DNA, 185 bp, Homo sapiens
ccctggcctgagggatcagcaggcactgatatgtctctctttgctctgaaccaacagactcttttcttcaacttgtttgactctcctggggagctgtttgatgagcccatctttatcacggtatgtctcagcagtcaaagtgttctccgtgggctgtatgtatgcacataggtgtcagtgcacac

SEQ ID NO: 39 - DNA, 196 bp, Homo sapiens
aagagctattgggttggccgtgtgggccacatgtccctgtgaatgtgagccatgatctttctctgcaggtggtagactctcgttctctcaggacagatgctctcctcggggagttccgggtaattgcttattttctaaaagcagtcagttctcacttctccgtgttggtggagcctctgtggaccatgggcagggg

SEQ ID NO: 40 - DNA, 178 bp, Homo sapiens
tggaatcgtataatgcaccacactttatttaacgctttggcggcaagagtttgatttgtgtctcctctcttgattgcagatggacgtgggcaccatttacagagagccccgtgagttctcaccactttggccgtatccttgcattttggttctggaggctgattggggacactcattt

SEQ ID NO: 41 - DNA, 231 bp, Homo sapiens
ggggtcttctgattctgggatcaccaaaggatgttgtctctcttagggcacgcctatctcaggaagtggctgctgctctcagaccctgatgacttctctgctggggccagaggctacctgaaaacaagcctttgtgtgctggggcctggggacgaagcgcctgtgagtacatttccctgggtcttccttacggtcccccacgcggcacttggttgcggaggcaccaaacca

SEQ ID NO: 42 - DNA, 247 bp, Homo sapiens
gtcaaaaccctgtgctcaggagcgcatgaaggaacgtatttggttttctttgtagctggagagaaaagacccctctgaagacaaggaggacattgaaagcaacctgctccggcccacaggcgtagccctgcgaggagcccacttctgcctgaaggtcttccgggccgaggacttgccgcagagtgcgtggggcgcgcccttgggtgggaggtctgcaggaggctggaggcgcagggctggtgggggt

SEQ ID NO: 43 - DNA, 179 bp, Homo sapiens
caggcagtgactggtgtgtccctcttcccagtggacgatgccgtgatggacaacgtgaaacagatctttggcttcgagagtaacaagaagaacttggtggacccctttgtggaggtcagctttgcggggaaaatggtaaggagcaagggagcaggagggttctctcgggaggggacggg

SEQ ID NO: 44 - DNA, 202 bp, Homo sapiens
ccccgggggagcccagagtccccatggagctgatcaacttgtcccctccctgtgtcttctagctgtgcagcaagatcttggagaagacggccaaccctcagtggaaccagaacatcacactgcctgccatggtgagcctcctgtccccagcaaacccaaggaggcccctggggctctgggcttcgggaggtccagggctcct

SEQ ID NO: 45 - DNA, 167 bp, Homo sapiens
gggaggggctgttctatcttcaaaaggactcttctcccaacacgcctctattccttcctcagtttccctccatgtgcgaaaaaatgaggattcgtatcatagactggtgagttctgagtcttggagtctttagggcgggctgtcctgagggggcgctccctcagttt

SEQ ID NO: 46 - DNA, 220 bp, Homo sapiens
tgtggcctgagttcctttcctgtgtcaggccctctctgctcccttgctctctagggaccgcctgactcacaatgacatcgtggctaccacctacctgagtatgtcgaaaatctctgcccctggaggagaaatagaaggtatgttccctcttcgttctgccctttgaccccctgtgctctccccccctctatccagcttacacttctagttttgagagttt

SEQ ID NO: 47 - DNA, 172 bp, Homo sapiens
acagcctgttcatgtaacccgtccttctcccagccatgcccaccctaaccccttttccatttctttacgcttcagaggagcctgcaggtgctgtcaagccttcgaaagcctcagactgtacgttgctgtcaccttggggacaaccaggggagtggggccttgggttttggct

SEQ ID NO: 48 - DNA, 200 bp, Homo sapiens
ccgacccctctgattgccacttgtgtctcccagtggatgactacctgggcttcctccccacttttgggccctgctacatcaacctctatggcagtcccagagagttcacaggcttcccagacccctacacagagctcaacacaggcaaggtaagccggctggagccctggcaagggcaggatgccacatgcccaggtggg

SEQ ID NO: 49 - DNA, 217 bp, Homo sapiens
cctcccctctgtctcccctgctccttgtgacctgacctccctggcagggggaaggtgtggcttatcgtggccggcttctgctctccctggagaccaagctggtggagcacagtgaacagaaggtggaggaccttcctgcggatgacatcctccgggtggaggtgaggggtgtggctctgggtgggagctgggcgtcggggcagggaagggatggcca

SEQ ID NO: 50 - DNA, 269 bp, Homo sapiens
agcctgggtgcctttctttgctcctcccgtgaccctctggtctactctctgctctcagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacggtgaggcagtgctcctggctgggaccccgatca

SEQ ID NO: 51 - DNA, 225 bp, Homo sapiens
actcctggcacagcgctcaggcccgtctctccattccagggtgccactactactacctaccctggggtaacgtgaaacctgtggtggtgctgtcatcctactgggaggacatcagccatagaatcgagactcagaaccagctgcttgggattgctgaccggctggtgagtgaaaacttgcccaaagctgcacatgcctatgcatgcacctgctacccccgctgca

SEQ ID NO: 52 - DNA, 227 bp, Homo sapiens
gggtccagcatgcaccctctgccctgtggtgacacacctgacccttgcctgcccattccacaggaagctggcctggagcaggtccacctggccctgaaggcgcagtgctccacggaggacgtggactcgctggtggctcagctgacggatgagctcatcgcaggctgcaggtaggggggacctggcgcccctggtgcccacctctcctggctcaactgggcctgttt

SEQ ID NO: 53 - DNA, 303 bp, Homo sapiens
tgggagaccctgggctcatcaggcgcattccatctgtccgtccctcacagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggaggtaattaagcctgggggtgcctttcttcttctgctctcctgctgcctggaacatcagaac

SEQ ID NO: 54 - DNA, 272 bp, Homo sapiens
cgtgggcctggtgtgtcaccatccccaccccgaccaccaccctctgttcagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaagtgagttttctttttccaagtcatgatcgtatttccaacataaggcctttctcccatctcttgct

SEQ ID NO: 55 - DNA, 219 bp, Homo sapiens
tgtgggtttctgtccttcttcggtacccagtatccgatggagaaggtgcctggcgcccggatgccagtgcagatacgggtcaagctgtggtttgggctctctgtggatgagaaggagttcaaccagtttgctgaggggaagctgtctgtctttgctgaaaccgtgagtacctgccagcccccacctctgcctcccactacctggagctgccttggcccc

SEQ ID NO: 56 - DNA, 292 bp, Homo sapiens
tgcctcccactacctggagctgccttggcccccttcacgcctcattcttcctggccctccagtatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagacgtgagtcgtgggcagggagggctggggagagccaggccaggctgcccaccatggactgcaccc

SEQ ID NO: 57 - DNA, 242 bp, Homo sapiens
tggatgggggcctctccagcagagcagcagagactctgaccagccctcctccacagtctgctccatgacatggacgccggtcacctgagcttcgtggaagaggtgtttgagaaccagacccggcttcccggaggccagtggatctacatgagtgacaactacaccgatgtggtaaagcaggcactcaggggcaggtggggtctagacatttggtctctggaggcacctggtgctcagggaca

SEQ ID NO: 58 - DNA, 215 bp, Homo sapiens
tcacatctgtctgtctcctctcattgcttgcctgttcggttttgtccttagaacggggagaaggtgcttcccaaggatgacattgagtgcccactgggctggaagtgggaagatgaggaatggtccacagacctcaaccgggctgtcgatgagcaaggtgggcagcatgtggaacctggcgagccccatccccggcaagctctcaagccatgcat

SEQ ID NO: 59 - DNA, 246 bp, Homo sapiens
agagatggtcccaggagagatggggggaagtgccaagcaatgagtgaccggttccccctcccccaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaagggtgagccagcaggtggtgggtgggagtgaggcctgt

SEQ ID NO: 60 - DNA, 253 bp, Homo sapiens
cttcccaccggcctctgagtctgccccttcttgtgcagcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctggtatgtggggctgcacttgtcctggcttgggtagggtatat

SEQ ID NO: 61 - DNA, 177 bp, Homo sapiens
gaatctgccataaccagcttcgtgtctccagggcggcgtgatggatgacaagagtgaagattccatgtccgtctccaccttgagcttcggtgtgaacagacccacgatttcctgcatattcgactgtaagtaggcttcgaggcctctatggggtgataagggtgtgtcaccttatgc

SEQ ID NO: 62 - DNA, 181 bp, Homo sapiens
aaccactccagccactcactctggcacctctgttttttcccttggtgaagatgggaaccgctaccatctacgctgctacatgtaccaggcccgggacctggctgcgatggacaaggactctttttctggtaggtgggagagaggcaggagagtcagagactgtgggctgagatctgggaat

SEQ ID NO: 63 - DNA, 319 bp, Homo sapiens
ccccacatggctctggagaagacatctctcagggtccctgctgtgtaatgtctcccctccccctctggccatgcagatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatgtgagtctgcccagctcctgcctcgtcccctcacagggagggaccatgtgcaaaggtgggg

SEQ ID NO: 64 - DNA, 249 bp, Homo sapiens
gccctgggtaagggatgctgattcttgtctctctacgcttggtctagggtgcagacgagtttatgggtcgctgcatctgtcaaccgagtctggaacggatgccacggctggcctggttcccactgacgaggggcagccagccgtcgggggagctgctggcctcttttgagctcatccagagagagaaggtgaggctggtctatatccagatccaggaggcccaggcaggagtggggtgggggccaaccc

SEQ ID NO: 65 - DNA, 158 bp, Homo sapiens
cactgacatagtccatgagtgtcatgagggtgatgggggccttaggtgacaagcacatgaccagagctctcttttcttcactccagccggccatccaccatattcctggttttgaggtaagtcttgctctgacctttccttcttcaaactgattgcca

SEQ ID NO: 66 - DNA, 132 bp, Homo sapiens
ctttttccccttccaacccctctcaccatctcctgatgtgcacatcccatggctgtgggccaggtgcaggagacatcaaggatcctggatgaggtgagctggcggggccgaggtagagggaaggtgaagcca

SEQ ID NO: 67 - DNA, 216 bp, Homo sapiens
tcttccttccacctttgtctccattctacctgctgtccactgcagtctgaggacacagacctgccctacccaccaccccagagggaggccaacatctacatggttcctcagaacatcaagccagcgctccagcgtaccgccatcgaggtgagccgtccgggcctgggcgtgggggctgggagcagcctgcccttccccttcctggccccagccttt

SEQ ID NO: 68 - DNA, 263 bp, Homo sapiens
cccgggccttctgagccactctcctcattctgtgtgcttagaatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtggtgagccccacctccctactgtccccttccagagtcctggggctagaagttctacatgt

SEQ ID NO: 69 - DNA, 249 bp, Homo sapiens
caggccagtgcgttcttcctcctccacccagatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccaggtaggggaaggggagatgatgggcaggtcagggaagggggagcctagggcaa

SEQ ID NO: 70 - DNA, 180 bp, Homo sapiens
aggggcgagccttttgagagagcccctgtcaggcctggatggctccctcccctgcagacgatgtgagcctactcagtcctggggaagacgtgctcatcgacattgatgacaaggagcccctcatccccatccaggtaggatgggcatcctccagggaggcctgggtcacctttcccctcc

SEQ ID NO: 71 - DNA, 211 bp, Homo sapiens
tgctgcttggcgagtcctgtttctgaaatggtctctttctttctacccactcaggaggaagagttcatcgattggtggagcaaattctttgcctccataggggagagggaaaagtgcggctcctacctggagaaggattttgacaccctgaaggtaaggcctctcttcagtctgacagtcggtgtgtgtgtgcgtgctgggcagtgggaga

SEQ ID NO: 72 - DNA, 235 bp, Homo sapiens
gttctactttctttctgtctcttgtcccctcctctaatccccatgtgtggcaggtctatgacacacagctggagaatgtggaggcctttgagggcctgtctgacttttgtaacaccttcaagctgtaccggggcaagacgcaggaggagacagaagatccatctgtgattggtgaatttaaggtaaatcctcgaagacgtccctaacccaggtgggcctaagactgtggtgttgg

SEQ ID NO: 73 - DNA, 268 bp, Homo sapiens
ggggacacagccaaaccatatcaacaatgatgataaaataaaattaacccttccttcttttcagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaaggtaactttctagagccctcacctccccagagtagcaggctcaggtaca

SEQ ID NO: 74 - DNA, 200 bp, Homo sapiens
tttggaaagtgttttcacagaagtgttttgtctcctcctccagtgtgatccttacatcaagatctccatagggaagaaatcagtgagtgaccaggataactacatcccctgcacgctggagcccgtatttggaaagtaaattggggcatcttgggtcttggggtggaggagccagacaggataacccacagtctagtggg

SEQ ID NO: 75 - DNA, 263 bp, Homo sapiens
cctgttcccttgggtgccctgtgttggctgacattcgggaatctgccccttcctgcaggatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtgtacgtggatgggggctggctgcctgcttctctg

SEQ ID NO: 76 - DNA, 237 bp, Homo sapiens
aagcatctcgtctatgtcttgtgcttgctcctcagctctggaccgaaccagtggcgggaccagctccgcccctcccagctcctccacctcttctgccagcagcatagagtcaaggcacctgtgtaccggacagaccgtgtaatgtttcaggataaagaatattccattgaagagataggtgagctgccacatgaccccaaaccatggtgggctctcgctgtatccctccctctctca

SEQ ID NO: 77 - DNA, 245 bp, Homo sapiens
tctctcgcttccccagctcctgcaacttttttgtgttctctctggggcagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggtaggaccttacccttggtcccagagtcctcgaactccagaagcccaaccccagg

SEQ ID NO: 78 - DNA, 214 bp, Homo sapiens
ggtgcttggtaacagctggttaaatgagaagggtggggagagaacggacctgtctccgcaggggaagctggggaagctgcagatgtgggtcgacctatttccgaaggccctggggcggcctggacctcccttcaacatcaccccacggagagccagaaggtgacttcccagccacaggctctgagctgggctgaggggtggggcgttgcagcct

SEQ ID NO: 79 - DNA, 229 bp, Homo sapiens
ttcttaaggccttcccatcctttggtaggaaatctaggtggattagagtgatacctttccccaggtttttcctgcgttgtattatctggaataccagagatgtgatcctggatgacctgagcctcacgggggagaagatgagcgacatttatgtgaaagggtagggagccagcgtcctcttgcctgtccagcttcccgcagctcccgtgctccctctgggttgtgcaca

SEQ ID NO: 80 - DNA, 261 bp, Homo sapiens
acgatgtatatactgtgttggaaatcttaatgagaactattctctaaaaacatgtatgtctagttggatgattggctttgaagaacacaagcaaaagacagacgtgcattatcgttccctgggaggtgaaggcaacttcaactggaggttcattttccccttcgactacctgccagctgagcaagtctgtaccattgccaagaaggtcagtgtccttccgattccctgtggtgccagcaccagggcttctaaagttagcct

SEQ ID NO: 81 - DNA, 234 bp, Homo sapiens
tgcctctctctaactttgcttccttgcatccttctctgttcctcttccgggtcaggatgccttctggaggctggacaagactgagagcaaaatcccagcacgagtggtgttccagatctgggacaatgacaagttctcctttgatgattttctggtgattttctgggtaagcgctattgctagaatcccattctgcacatgggggctgccccagaacccacactgtgtgtttat

SEQ ID NO: 82 - DNA, 297 bp, Homo sapiens
ggctacaggctggcagtgatcgagaaacccggccaaaaaccacctctctgttgcaggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcggtaagtctacttcctccagccccagtggagggcatgggggaagcttcttccatagaaattgt

SEQ ID NO: 83 - DNA, 237 bp, Homo sapiens
cctggttactctccaggccactgagcagagccttcgtgcccctaaccaagtgctctctgtcccctcagggcaagctggaaatgaccttggagattgtagcagagagtgagcatgaggagcggcctgctggccagggccgggatgagcccaacatgaaccctaagcttgaggacccaaggtcagtgcccagcccctgagccccaatgcccacaggtctgggggtataggcacagtcca

SEQ ID NO: 84 - DNA, 252 bp, Homo sapiens
ccctagtaaaggatgcccagttgactccgggatctcgcttccaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccggtgagcaggcctgacgacactgtggtgggggaactctgggtctaatgggggagttcatca

SEQ ID NO: 85 - DNA, 391 bp, Homo sapiens
tggctgtgcctgccccagtgggatcaccatgggtccctgtctcctccctccctccagaactatgctgccatgaagctggtgaagcccttcagctgaggactctcctgccctgtagaaggggccgtggggtcccctccagcatgggactggcctgcctcctccgcccagctcggcgagctcctccagacctcctaggcctgattgtcctgccagggtgggcagacagacagatggaccggcccacactcccagagttgctaacatggagctctgagatcaccccacttccatcatttccttctcccccaacccaacgcttttttggatcagctcagacatatttcagtataaaacagttggaaccacaaaaaaaaaaaaaaaaaaaaaaaaa

SEQ ID NO: 86 - protein, 51 aa, Homo sapiens
KKRTKVIKNSVNPVWNEGFEWDLKGIPLDQGSELHVVVKDHETMGRNRFLG

SEQ ID NO: 87 - protein, 45 aa, Homo sapiens
SKILEKTANPQWNQNITLPAMFPSMCEKMRIRIIDWDRLTHNDIV

SEQ ID NO: 88 - protein, 82 aa, Homo sapiens
QARDLAAMDKDSFSDPYAIVSFLHQSQKTVVVKNTLNPTWDQTLIFYEIEIFGEPATVAEQPPSIVVELYDHDTYGADEFMG

SEQ ID NO: 89 - protein, 79 aa, Homo sapiens
IYIVRAFGLQPKDPNGKCDPYIKISIGKKSVSDQDNYIPCTLEPVFGKMFELTCTLPLEKDLKITLYDYDLLSKDEKIG

SEQ ID NO: 90 - DNA, 152 bp, Homo sapiens
acgatgtatatactgtgttggaaatcttaatgagaactattctctaaaaacatgtatgtctagttggatgattggctttgaagaacacaagcaaaagacagacgtgcattatcgttccctgggaggtgaaggcaacttcaactggaggttca

SEQ ID NO: 91 - DNA, 56 bp, Homo sapiens
gtcagtgtccttccgattccctgtggtgccagcaccagggcttctaaagttagcct

SEQ ID NO: 92 - DNA, 55 bp, Homo sapiens
tgcctctctctaactttgcttccttgcatccttctctgttcctcttccgggtcag

SEQ ID NO: 93 - DNA, 68 bp, Homo sapiens
gtaagcgctattgctagaatcccattctgcacatgggggctgccccagaacccacactgtgtgtttat

SEQ ID NO: 94 - DNA, 56 bp, Homo sapiens
ggctacaggctggcagtgatcgagaaacccggccaaaaaccacctctctgttgcag

SEQ ID NO: 95 - DNA, 62 bp, Homo sapiens
gtaagtctacttcctccagccccagtggagggcatgggggaagcttcttccatagaaattgt

SEQ ID NO: 96 - DNA, 68 bp, Homo sapiens
cctggttactctccaggccactgagcagagccttcgtgcccctaaccaagtgctctctgtcccctcag

SEQ ID NO: 97 - DNA, 59 bp, Homo sapiens
gtcagtgcccagcccctgagccccaatgcccacaggtctgggggtataggcacagtcca

SEQ ID NO: 98 - DNA, 44 bp, Homo sapiens
ccctagtaaaggatgcccagttgactccgggatctcgcttccag

SEQ ID NO: 99 - DNA, 60 bp, Homo sapiens
gtgagcaggcctgacgacactgtggtgggggaactctgggtctaatgggggagttcatca

SEQ ID NO: 100 - DNA, 57 bp, Homo sapiens
tggctgtgcctgccccagtgggatcaccatgggtccctgtctcctccctccctccag

SEQ ID NO: 101 - DNA, 23 bp, Homo sapiens
tctcttctcctagagggccatag

SEQ ID NO: 102 - DNA, 24 bp, Homo sapiens
ctgttcctccccatcgtctcatgg

SEQ ID NO: 103 - DNA, 20 bp, Homo sapiens
gctcctcccgtgaccctctg

SEQ ID NO: 104 - DNA, 21 bp, Homo sapiens
gggtcccagccaggagcactg

SEQ ID NO: 105 - DNA, 24 bp, Homo sapiens
cccctctcaccatctcctgatgtg

SEQ ID NO: 106 - DNA, 25 bp, Homo sapiens
tggcttcaccttccctctacctcgg

SEQ ID NO: 107 - DNA, 24 bp, Homo sapiens
tcctttggtaggaaatctaggtgg

SEQ ID NO: 108 - DNA, 21 bp, Homo sapiens
ggaagctggacaggcaagagg

SEQ ID NO: 109 - DNA, 27 bp, Homo sapiens
atatactgtgttggaaatcttaatgag

SEQ ID NO: 110 - DNA, 21 bp, Homo sapiens
gctggcaccacagggaatcgg

SEQ ID NO: 111 - DNA, 25 bp, Homo sapiens
ctttgcttccttgcatccttctctg

SEQ ID NO: 112 - DNA, 21 bp, Homo sapiens
agcccccatgtgcagaatggg

SEQ ID NO: 113 - DNA, 21 bp, Homo sapiens
ggcagtgatcgagaaacccgg

SEQ ID NO: 114 - DNA, 21 bp, Homo sapiens
catgccctccactggggctgg

SEQ ID NO: 115 - DNA, 21 bp, Homo sapiens
ggatgcccagttgactccggg

SEQ ID NO: 116 - DNA, 21 bp, Homo sapiens
ccccaccacagtgtcgtcagg

SEQ ID NO: 117 - DNA, 6240 bp, Homo sapiens
atgctgagggtcttcatcctctatgccgagaacgtccacacacccgacaccgacatcagcgatgcctactgctccgcggtgtttgcaggggtgaagaagagaaccaaagtcatcaagaacagcgtgaaccctgtatggaatgagggatttgaatgggacctcaagggcatccccctggaccagggctctgagcttcatgtggtggtcaaagaccatgagacgatggggaggaacaggttcctgggggaagccaaggtcccactccgagaggtcctcgccacccctagtctgtccgccagcttcaatgcccccctgctggacaccaagaagcagcccacaggggcctcgctggtcctgcaggtgtcctacacaccgctgcctggagctgtgcccctgttcccgccccctactcctctggagccctccccgactctgcctgacctggatgtagtggcagacacaggaggagaggaagacacagaggaccagggactcactggagatgaggcggagccattcctggatcaaagcggaggcccgggggctcccaccaccccaaggaaactaccttcacgtcctccgccccactaccccgggatcaaaagaaagcgaagtgcgcctacatctagaaagctgctgtcagacaaaccgcaggatttccagatcagggtccaggtgatcgaggggcgccagctgccgggggtgaacatcaagcctgtggtcaaggttaccgctgcagggcagaccaagcggacgcggatccacaagggaaacagcccactcttcaatgagactcttttcttcaacttgtttgactctcctggggagctgtttgatgagcccatctttatcacggtggtagactctcgttctctcaggacagatgctctcctcggggagttccggatggacgtgggcaccatttacagagagccccggcacgcctatctcaggaagtggctgctgctctcagaccctgatgacttctctgctggggccagaggctacctgaaaacaagcctttgtgtgctggggcctggggacgaagcgcctctggagagaaaagacccctctgaagacaaggaggacattgaaagcaacctgctccggcccacaggcgtagccctgcgaggagcccacttctgcctgaaggtcttccgggccgaggacttgccgcagatggacgatgccgtgatggacaacgtgaaacagatctttggcttcgagagtaacaagaagaacttggtggacccctttgtggaggtcagctttgcggggaaaatgctgtgcagcaagatcttggagaagacggccaaccctcagtggaaccagaacatcacactgcctgccatgtttccctccatgtgcgaaaaaatgaggattcgtatcatagactgggaccgcctgactcacaatgacatcgtggctaccacctacctgagtatgtcgaaaatctctgcccctggaggagaaatagaagaggagcctgcaggtgctgtcaagccttcgaaagcctcagacttggatgactacctgggcttcctccccacttttgggccctgctacatcaacctctatggcagtcccagagagttcacaggcttcccagacccctacacagagctcaacacaggcaagggggaaggtgtggcttatcgtggccggcttctgctctccctggagaccaagctggtggagcacagtgaacagaaggtggaggaccttcctgcggatgacatcctccgggtggagaagtaccttaggaggcgcaagtactccctgtttgcggccttctactcagccaccatgctgcaggatgtggatgatgccatccagtttgaggtcagcatcgggaactacgggaacaagttcgacatgacctgcctgccgctggcctccaccactcagtacagccgtgcagtctttgacgggtgccactactactacctaccctggggtaacgtgaaacctgtggtggtgctgtcatcctactgggaggacatcagccatagaatcgagactcagaaccagctgcttgggattgctgaccggctggaagctggcctggagcaggtccacctggccctgaaggcgcagtgctccacggaggacgtggactcgctggtggctcagctgacggatgagctcatcgcaggctgcagccagcctctgggtgacatccatgagacaccctctgccacccacctggaccagtacctgtaccagctgcgcacccatcacctgagccaaatcactgaggctgccctggccctgaagctcggccacagtgagctccctgcagctctggagcaggcggaggactggctcctgcgtctgcgtgccctggcagaggagccccagaacagcctgccggacatcgtcatctggatgctgcagggagacaagcgtgtggcataccagcgggtgcccgcccaccaagtcctcttctcccggcggggtgccaactactgtggcaagaattgtgggaagctacagacaatctttctgaaatatccgatggagaaggtgcctggcgcccggatgccagtgcagatacgggtcaagctgtggtttgggctctctgtggatgagaaggagttcaaccagtttgctgaggggaagctgtctgtctttgctgaaacctatgagaacgagactaagttggcccttgttgggaactggggcacaacgggcctcacctaccccaagttttctgacgtcacgggcaagatcaagctacccaaggacagcttccgcccctcggccggctggacctgggctggagattggttcgtgtgtccggagaagactctgctccatgacatggacgccggtcacctgagcttcgtggaagaggtgtttgagaaccagacccggcttcccggaggccagtggatctacatgagtgacaactacaccgatgtgaacggggagaaggtgcttcccaaggatgacattgagtgcccactgggctggaagtgggaagatgaggaatggtccacagacctcaaccgggctgtcgatgagcaaggctgggagtatagcatcaccatccccccggagcggaagccgaagcactgggtccctgctgagaagatgtactacacacaccgacggcggcgctgggtgcgcctgcgcaggagggatctcagccaaatggaagcactgaaaaggcacaggcaggcggaggcggagggcgagggctgggagtacgcctctctttttggctggaagttccacctcgagtaccgcaagacagatgccttccgccgccgccgctggcgccgtcgcatggagccactggagaagacggggcctgcagctgtgtttgcccttgagggggccctgggcggcgtgatggatgacaagagtgaagattccatgtccgtctccaccttgagcttcggtgtgaacagacccacgatttcctgcatattcgactatgggaaccgctaccatctacgctgctacatgtaccaggcccgggacctggctgcgatggacaaggactctttttctgatccctatgccatcgtctccttcctgcaccagagccagaagacggtggtggtgaagaacacccttaaccccacctgggaccagacgctcatcttctacgagatcgagatctttggcgagccggccacagttgctgagcaaccgcccagcattgtggtggagctgtacgaccatgacacttatggtgcagacgagtttatgggtcgctgcatctgtcaaccgagtctggaacggatgccacggctggcctggttcccactgacgaggggcagccagccgtcgggggagctgctggcctcttttgagctcatccagagagagaagccggccatccaccatattcctggttttgaggtgcaggagacatcaaggatcctggatgagtctgaggacacagacctgccctacccaccaccccagagggaggccaacatctacatggttcctcagaacatcaagccagcgctccagcgtaccgccatcgagatcctggcatggggcctgcggaacatgaagagttaccagctggccaacatctcctcccccagcctcgtggtagagtgtgggggccagacggtgcagtcctgtgtcatcaggaacctccggaagaaccccaactttgacatctgcaccctcttcatggaagtgatgctgcccagggaggagctctactgcccccccatcaccgtcaaggtcatcgataaccgccagtttggccgccggcctgtggtgggccagtgtaccatccgctccctggagagcttcctgtgtgacccctactcggcggagagtccatccccacagggtggcccagacgatgtgagcctactcagtcctggggaagacgtgctcatcgacattgatgacaaggagcccctcatccccatccaggaggaagagttcatcgattggtggagcaaattctttgcctccataggggagagggaaaagtgcggctcctacctggagaaggattttgacaccctgaaggtctatgacacacagctggagaatgtggaggcctttgagggcctgtctgacttttgtaacaccttcaagctgtaccggggcaagacgcaggaggagacagaagatccatctgtgattggtgaatttaagggcctcttcaaaatttatcccctcccagaagacccagccatccccatgcccccaagacagttccaccagctggccgcccagggaccccaggagtgcttggtccgtatctacattgtccgagcatttggcctgcagcccaaggaccccaatggaaagtgtgatccttacatcaagatctccatagggaagaaatcagtgagtgaccaggataactacatcccctgcacgctggagcccgtatttggaaagatgttcgagctgacctgcactctgcctctggagaaggacctaaagatcactctctatgactatgacctcctctccaaggacgaaaagatcggtgagacggtcgtcgacctggagaacaggctgctgtccaagtttggggctcgctgtggactcccacagacctactgtgtctctggaccgaaccagtggcgggaccagctccgcccctcccagctcctccacctcttctgccagcagcatagagtcaaggcacctgtgtaccggacagaccgtgtaatgtttcaggataaagaatattccattgaagagatagaggctggcaggatcccaaacccacacctgggcccagtggaggagcgtctggctctgcatgtgcttcagcagcagggcctggtcccggagcacgtggagtcacggcccctctacagccccctgcagccagacatcgagcaggggaagctgcagatgtgggtcgacctatttccgaaggccctggggcggcctggacctcccttcaacatcaccccacggagagccagaaggtttttcctgcgttgtattatctggaataccagagatgtgatcctggatgacctgagcctcacgggggagaagatgagcgacatttatgtgaaaggttggatgattggctttgaagaacacaagcaaaagacagacgtgcattatcgttccctgggaggtgaaggcaacttcaactggaggttcattttccccttcgactacctgccagctgagcaagtctgtaccattgccaagaaggatgccttctggaggctggacaagactgagagcaaaatcccagcacgagtggtgttccagatctgggacaatgacaagttctcctttgatgattttctgggctccctgcagctcgatctcaaccgcatgcccaagccagccaagacagccaagaagtgctccttggaccagctggatgatgctttccacccagaatggtttgtgtccctttttgagcagaaaacagtgaagggctggtggccctgtgtagcagaagagggtgagaagaaaatactggcgggcaagctggaaatgaccttggagattgtagcagagagtgagcatgaggagcggcctgctggccagggccgggatgagcccaacatgaaccctaagcttgaggacccaaggcgccccgacacctccttcctgtggtttacctccccatacaagaccatgaagttcatcctgtggcggcgtttccggtgggccatcatcctcttcatcatcctcttcatcctgctgctgttcctggccatcttcatctacgccttcccgaactatgctgccatgaagctggtgaagcccttcagc

SEQ ID NO: 118 - DNA, 13 bp, Homo sapiens
cgcaagcatgctg

SEQ ID NO: 119 - DNA, 12 bp, Homo sapiens
gagacgatgggg

SEQ ID NO: 120 - DNA, 21 bp, Homo sapiens
gatctaaccctgctgctcacc

SEQ ID NO: 121 - DNA, 21 bp, Homo sapiens
ctggtgtgttgcagagcgctg

SEQ ID NO: 122 - DNA, 21 bp, Homo sapiens
cctctcttctgctgtcttcag

SEQ ID NO: 123 - DNA, 21 bp, Homo sapiens
tgtgtctggttcaccttcgtg

SEQ ID NO: 124 - DNA, 21 bp, Homo sapiens
tccaaatagaaatgcctgaac

SEQ ID NO: 125 - DNA, 21 bp, Homo sapiens
aggtatcacctccaagtgttg

SEQ ID NO: 126 - DNA, 21 bp, Homo sapiens
taccagcttcagagctccctg

SEQ ID NO: 127 - DNA, 19 bp, Homo sapiens
ttgatcagggtgctcttgg

SEQ ID NO: 128 - DNA, 20 bp, Homo sapiens
ggagaattgcttgaacccag

SEQ ID NO: 129 - DNA, 22 bp, Homo sapiens
tggctaatgatgttgaacattt

SEQ ID NO: 130 - DNA, 21 bp, Homo sapiens
gacccacaagcggcgcctcgg

SEQ ID NO: 131 - DNA, 21 bp, Homo sapiens
gaccccggcgagggtggtcgg

SEQ ID NO: 132 - DNA, 24 bp, Homo sapiens
tgtctctccattctcccttttgtg

SEQ ID NO: 133 - DNA, 24 bp, Homo sapiens
aggacactgctgagaaggcacctc

SEQ ID NO: 134 - DNA, 21 bp, Homo sapiens
agtgccctggtggcacgaagg

SEQ ID NO: 135 - DNA, 24 bp, Homo sapiens
cctacctgcaccttcaagccatgg

SEQ ID NO: 136 - DNA, 23 bp, Homo sapiens
cagaagagccagggtgccttagg

SEQ ID NO: 137 - DNA, 24 bp, Homo sapiens
ccttggaccttaacctggcagagg

SEQ ID NO: 138 - DNA, 21 bp, Homo sapiens
cgaggccagcgcaccaacctg

SEQ ID NO: 139 - DNA, 22 bp, Homo sapiens
actgccggccattcttgctggg

SEQ ID NO: 140 - DNA, 21 bp, Homo sapiens
ccaggcctcattagggccctc

SEQ ID NO: 141 - DNA, 22 bp, Homo sapiens
ctgaagaggagcctggggtcag

SEQ ID NO: 142 - DNA, 24 bp, Homo sapiens
ctgagatttctgactcttggggtg

SEQ ID NO: 143 - DNA, 24 bp, Homo sapiens
aaggttctgccctcatgccccatg

SEQ ID NO: 144 - DNA, 21 bp, Homo sapiens
ctggcctgagggatcagcagg

SEQ ID NO: 145 - DNA, 23 bp, Homo sapiens
gtgcatacatacagcccacggag

SEQ ID NO: 146 - DNA, 24 bp, Homo sapiens
gagctattgggttggccgtgtggg

SEQ ID NO: 147 - DNA, 24 bp, Homo sapiens
accaacacggagaagtgagaactg

SEQ ID NO: 148 - DNA, 26 bp, Homo sapiens
ccacactttatttaacgctttggcgg

SEQ ID NO: 149 - DNA, 24 bp, Homo sapiens
cagaaccaaaatgcaaggatacgg

SEQ ID NO: 150 - DNA, 25 bp, Homo sapiens
cttctgattctgggatcaccaaagg

SEQ ID NO: 151 - DNA, 22 bp, Homo sapiens
ggaccgtaaggaagacccaggg

SEQ ID NO: 152 - DNA, 24 bp, Homo sapiens
cctgtgctcaggagcgcatgaagg

SEQ ID NO: 153 - DNA, 22 bp, Homo sapiens
gcagacctcccacccaagggcg

SEQ ID NO: 154 - DNA, 24 bp, Homo sapiens
gagacagatgggggacagtcaggg

SEQ ID NO: 155 - DNA, 21 bp, Homo sapiens
cctcccgagagaaccctcctg

SEQ ID NO: 156 - DNA, 21 bp, Homo sapiens
gggagcccagagtccccatgg

SEQ ID NO: 157 - DNA, 21 bp, Homo sapiens
gggcctccttgggtttgctgg

SEQ ID NO: 158 - DNA, 21 bp, Homo sapiens
gcctccccagcatcctgccgg

SEQ ID NO: 159 - DNA, 24 bp, Homo sapiens
tcactgagccgaatgaaactgagg

SEQ ID NO: 160 - DNA, 24 bp, Homo sapiens
tgtggcctgagttcctttcctgtg

SEQ ID NO: 161 - DNA, 24 bp, Homo sapiens
ggtcaaagggcagaacgaagaggg

SEQ ID NO: 162 - DNA, 21 bp, Homo sapiens
cccgtccttctcccagccatg

SEQ ID NO: 163 - DNA, 21 bp, Homo sapiens
ctcccctggttgtccccaagg

SEQ ID NO: 164 - DNA, 24 bp, Homo sapiens
cgacccctctgattgccacttgtg

SEQ ID NO: 165 - DNA, 21 bp, Homo sapiens
ggcatcctgcccttgccaggg

SEQ ID NO: 166 - DNA, 20 bp, Homo sapiens
tctgtctcccctgctccttg

SEQ ID NO: 167 - DNA, 21 bp, Homo sapiens
cttccctgccccgacgcccag

SEQ ID NO: 168 - DNA, 21 bp, Homo sapiens
cagcgctcaggcccgtctctc

SEQ ID NO: 169 - DNA, 24 bp, Homo sapiens
tgcataggcatgtgcagctttggg

SEQ ID NO: 170 - DNA, 21 bp, Homo sapiens
catgcaccctctgccctgtgg

SEQ ID NO: 171 - DNA, 21 bp, Homo sapiens
agttgagccaggagaggtggg

SEQ ID NO: 172 - DNA, 24 bp, Homo sapiens
catcaggcgcattccatctgtccg

SEQ ID NO: 173 - DNA, 24 bp, Homo sapiens
agcaggagagcagaagaagaaagg

SEQ ID NO: 174 - DNA, 22 bp, Homo sapiens
gtgtgtcaccatccccaccccg

SEQ ID NO: 175 - DNA, 25 bp, Homo sapiens
caagagatgggagaaaggccttatg

SEQ ID NO: 176 - DNA, 23 bp, Homo sapiens
ctgggacatccggatcctgaagg

SEQ ID NO: 177 - DNA, 22 bp, Homo sapiens
tccaggtagtgggaggcagagg

SEQ ID NO: 178 - DNA, 24 bp, Homo sapiens
tcccactacctggagctgccttgg

SEQ ID NO: 179 - DNA, 21 bp, Homo sapiens
ggctctccccagccctccctg

SEQ ID NO: 180 - DNA, 24 bp, Homo sapiens
cagagcagcagagactctgaccag

SEQ ID NO: 181 - DNA, 21 bp, Homo sapiens
tagaccccacctgcccctgag

SEQ ID NO: 182 - DNA, 24 bp, Homo sapiens
tcctctcattgcttgcctgttcgg

SEQ ID NO: 183 - DNA, 21 bp, Homo sapiens
ttgagagcttgccggggatgg

SEQ ID NO: 184 - DNA, 24 bp, Homo sapiens
aagtgccaagcaatgagtgaccgg

SEQ ID NO: 185 - DNA, 21 bp, Homo sapiens
ctcactcccacccaccacctg

SEQ ID NO: 186 - DNA, 21 bp, Homo sapiens
cccaccggcctctgagtctgc

SEQ ID NO: 187 - DNA, 24 bp, Homo sapiens
accctacccaagccaggacaagtg

SEQ ID NO: 188 - DNA, 24 bp, Homo sapiens
gaatctgccataaccagcttcgtg

SEQ ID NO: 189 - DNA, 24 bp, Homo sapiens
tatcaccccatagaggcctcgaag

SEQ ID NO: 190 - DNA, 24 bp, Homo sapiens
cagccactcactctggcacctctg

SEQ ID NO: 191 - DNA, 24 bp, Homo sapiens
agcccacagtctctgactctcctg

SEQ ID NO: 192 - DNA, 24 bp, Homo sapiens
acatctctcagggtccctgctgtg

SEQ ID NO: 193 - DNA, 21 bp, Homo sapiens
cctgtgaggggacgaggcagg

SEQ ID NO: 194 - DNA, 24 bp, Homo sapiens
gccctgggtaagggatgctgattc

SEQ ID NO: 195 - DNA, 21 bp, Homo sapiens
cctgcctgggcctcctggatc

SEQ ID NO: 196 - DNA, 21 bp, Homo sapiens
gagggtgatgggggccttagg

SEQ ID NO: 197 - DNA, 24 bp, Homo sapiens
gcaatcagtttgaagaaggaaagg

SEQ ID NO: 198 - DNA, 24 bp, Homo sapiens
cacctttgtctccattctacctgc

SEQ ID NO: 199 - DNA, 21 bp, Homo sapiens
ctcccagcccccacgcccagg

SEQ ID NO: 200 - DNA, 24 bp, Homo sapiens
ctgagccactctcctcattctgtg

SEQ ID NO: 201 - DNA, 21 bp, Homo sapiens
tggaaggggacagtagggagg

SEQ ID NO: 202 - DNA, 22 bp, Homo sapiens
ggccagtgcgttcttcctcctc

SEQ ID NO: 203 - DNA, 22 bp, Homo sapiens
tccctgacctgcccatcatctc

SEQ ID NO: 204 - DNA, 21 bp, Homo sapiens
gcccctgtcaggcctggatgg

SEQ ID NO: 205 - DNA, 21 bp, Homo sapiens
tgacccaggcctccctggagg

SEQ ID NO: 206 - DNA, 24 bp, Homo sapiens
ctgaaatggtctctttctttctac

SEQ ID NO: 207 - DNA, 24 bp, Homo sapiens
cacaccgactgtcagactgaagag

SEQ ID NO: 208 - DNA, 24 bp, Homo sapiens
ttgtcccctcctctaatccccatg

SEQ ID NO: 209 - DNA, 21 bp, Homo sapiens
gggttagggacgtcttcgagg

SEQ ID NO: 210 - DNA, 22 bp, Homo sapiens
cagccaaaccatatcaacaatg

SEQ ID NO: 211 - DNA, 21 bp, Homo sapiens
ctggggaggtgagggctctag

SEQ ID NO: 212 - DNA, 21 bp, Homo sapiens
gaagtgttttgtctcctcctc

SEQ ID NO: 213 - DNA, 20 bp, Homo sapiens
gcaggcagccagcccccatc

SEQ ID NO: 214 - DNA, 21 bp, Homo sapiens
gggtgccctgtgttggctgac

SEQ ID NO: 215 - DNA, 20 bp, Homo sapiens
gcaggcagccagcccccatc

SEQ ID NO: 216 - DNA, 24 bp, Homo sapiens
ctcgtctatgtcttgtgcttgctc

SEQ ID NO: 217 - DNA, 23 bp, Homo sapiens
caccatggtttggggtcatgtgg

SEQ ID NO: 218 - DNA, 21 bp, Homo sapiens
tctcgcttccccagctcctgc

SEQ ID NO: 219 - DNA, 22 bp, Homo sapiens
tctggagttcgaggactctggg

SEQ ID NO: 220 - DNA, 21 bp, Homo sapiens
agaagggtggggagagaacgg

SEQ ID NO: 221 - DNA, 21 bp, Homo sapiens
cagctcagagcctgtggctgg

SEQ ID NO: 222 - DNA, 24 bp, Homo sapiens
aaggccttcccatcctttggtagg

SEQ ID NO: 223 - DNA, 21 bp, Homo sapiens
acaacccagagggagcacggg

SEQ ID NO: 224 - DNA, 25 bp, Homo sapiens
gttgacgatgtatatactgtgttgg

SEQ ID NO: 225 - DNA, 25 bp, Homo sapiens
gcctctctctaactttgcttccttg

SEQ ID NO: 226 - DNA, 24 bp, Homo sapiens
ggctacaggctggcagtgatcgag

SEQ ID NO: 227 - DNA, 21 bp, Homo sapiens
ttcccccatgccctccactgg

SEQ ID NO: 228 - DNA, 24 bp, Homo sapiens
agccttcgtgcccctaaccaagtg

SEQ ID NO: 229 - DNA, 21 bp, Homo sapiens
ctgtgggcattggggctcagg

SEQ ID NO: 230 - DNA, 20 bp, Homo sapiens
gccccagtgggatcaccatg

SEQ ID NO: 231 - DNA, 21 bp, Homo sapiens
atgctggaggggaccccacgg

SEQ ID NO: 232 - DNA, 20 bp, Homo sapiens
gcaggtgcgtgggatggacg

SEQ ID NO: 233 - DNA, 20 bp, Homo sapiens
catatcctcttcatccctgc

SEQ ID NO: 234 - DNA, 20 bp, Homo sapiens
gcccagccaggtgcaaaatg

SEQ ID NO: 235 - DNA, 20 bp, Homo sapiens
caaagagggctcggaaaggt

SEQ ID NO: 236 - DNA, 20 bp, Homo sapiens
acccacaagcggcgcctcgg

SEQ ID NO: 237 - DNA, 20 bp, Homo sapiens
tcggagtgggaccttggctt

SEQ ID NO: 238 - DNA, 23 bp, Homo sapiens
gctctgagcttcatgtggtggtc

SEQ ID NO: 239 - DNA, 22 bp, Homo sapiens
ccaggaatggctccgcctcatc

SEQ ID NO: 240 - DNA, 22 bp, Homo sapiens
ctctgcctgacctggatgtagt

SEQ ID NO: 241 - DNA, 22 bp, Homo sapiens
agtctcattgaagagtgggctg

SEQ ID NO: 242 - DNA, 23 bp, Homo sapiens
catcaagcctgtggtcaaggtta

SEQ ID NO: 243 - DNA, 23 bp, Homo sapiens
cttttctctccagaggcgcttcg

SEQ ID NO: 244 - DNA, 18 bp, Homo sapiens
tgctggggccagaggcta

SEQ ID NO: 245 - DNA, 19 bp, Homo sapiens
gttctggttccactgaggg

SEQ ID NO: 246 - DNA, 17 bp, Homo sapiens
gtggaggtcagctttgc

SEQ ID NO: 247 - DNA, 18 bp, Homo sapiens
ctgggaagcctgtgaact

SEQ ID NO: 248 - DNA, 19 bp, Homo sapiens
gcttcctccccacttttgg

SEQ ID NO: 249 - DNA, 19 bp, Homo sapiens
cggcaggcaggtcatgtcg

SEQ ID NO: 250 - DNA, 21 bp, Homo sapiens
gaggtcagcatcgggaactac

SEQ ID NO: 251 - DNA, 21 bp, Homo sapiens
ctggctgcagcctgcgatgag

SEQ ID NO: 252 - DNA, 21 bp, Homo sapiens
ctccacggaggacgtggactc

SEQ ID NO: 253 - DNA, 22 bp, Homo sapiens
cacccgctggtatgccacacgc

SEQ ID NO: 254 - DNA, 20 bp, Homo sapiens
cagaacagcctgccggacat

SEQ ID NO: 255 - DNA, 20 bp, Homo sapiens
cttggggtaggtgaggcccg

SEQ ID NO: 256 - DNA, 20 bp, Homo sapiens
cgagactaagttggcccttg

SEQ ID NO: 257 - DNA, 21 bp, Homo sapiens
aggtctgtggaccattcctca

SEQ ID NO: 258 - DNA, 21 bp, Homo sapiens
cttcccaaggatgacattgag

SEQ ID NO: 259 - DNA, 20 bp, Homo sapiens
tctccagtggctccatgcga

SEQ ID NO: 260 - DNA, 19 bp, Homo sapiens
ctcgagtaccgcaagacag

SEQ ID NO: 261 - DNA, 20 bp, Homo sapiens
cccaggtggggttaagggtg

SEQ ID NO: 262 - DNA, 19 bp, Homo sapiens
tggacaaggactctttttc

SEQ ID NO: 263 - DNA, 19 bp, Homo sapiens
cctcaaaaccaggaatatg

SEQ ID NO: 264 - DNA, 22 bp, Homo sapiens
gagctcatccagagagagaagc

SEQ ID NO: 265 - DNA, 20 bp, Homo sapiens
cacttccatgaagagggtgc

SEQ ID NO: 266 - DNA, 20 bp, Homo sapiens
gtgcagtcctgtgtcatcag

SEQ ID NO: 267 - DNA, 19 bp, Homo sapiens
gctccaccaatcgatgaac

SEQ ID NO: 268 - DNA, 20 bp, Homo sapiens
gagaagacgtgctcatcgac

SEQ ID NO: 269 - DNA, 20 bp, Homo sapiens
cagctggtggaactgtcttg

SEQ ID NO: 270 - DNA, 22 bp, Homo sapiens
gaagatccatctgtgattggtg

SEQ ID NO: 271 - DNA, 22 bp, Homo sapiens
ccttggagaggaggtcatagtc

SEQ ID NO: 272 - DNA, 21 bp, Homo sapiens
gatgttcgagctgacctgcac

SEQ ID NO: 273 - DNA, 20 bp, Homo sapiens
gtgtgggtttgggatcctgc

SEQ ID NO: 274 - DNA, 20 bp, Homo sapiens
ggcacctgtgtaccggacag

SEQ ID NO: 275 - DNA, 19 bp, Homo sapiens
ggatcacatctctggtatt

SEQ ID NO: 276 - DNA, 19 bp, Homo sapiens
gtgggtcgacctatttccg

SEQ ID NO: 277 - DNA, 19 bp, Homo sapiens
cagcctccagaaggcatcc

SEQ ID NO: 278 - DNA, 22 bp, Homo sapiens
gaggttcattttccccttcgac

SEQ ID NO: 279 - DNA, 21 bp, Homo sapiens
cagtattttcttctcaccctc

SEQ ID NO: 280 - DNA, 21 bp, Homo sapiens
gtccctttttgagcagaaaac

SEQ ID NO: 281 - DNA, 20 bp, Homo sapiens
cttcatggcagcatagttcg

SEQ ID NO: 282 - DNA, 20 bp, Homo sapiens
cttcatcctgctgctgttcc

SEQ ID NO: 283 - DNA, 21 bp, Homo sapiens
gtggttccaactgttttatac

(19) United States
(12) Patent Application Publication
(10) Pub. No.: US 2003/0110526 A1
(43) Pub. Date: Jun. 12, 2003

(54) DYSFERLIN MUTATIONS

(76) Inventors: ROBERT H. BROWN JR., NEEDHAM, MA (US); JING LIU, QUEBEC (CA); MASASHI AOKI, SENDAI (JP); ERIC HOFFMAN, KENSINGTON, MD (US); FAN-LI CHOU, PITTSBURG, PA (US)

Correspondence Address: FISH & RICHARDSON PC, 225 FRANKLIN ST, BOSTON, MA, 02110, US

(21) Appl. No.: 09/382,860

(22) Filed: Aug. 25, 1999

Related U.S. Application Data
(60) Provisional application No. 60/097,930, filed on Aug. 25, 1998.

(51) Int. Cl. A01H 1/00; C12Q 1/68; C07H 21/04; C12N 15/82; C12N 5/04; C12P 21/02

(52) U.S. Cl. 800/278; 435/6; 435/69.1; 435/320.1; 435/419; 536/23.2

(57) ABSTRACT

Mutations identified in dysferlin and methods to detect these mutations are described.

### RELATED APPLICATION INFORMATION

This application claims priority from provisional application serial No. 60/097,930, filed Aug. 25, 1998.

Statement as to Federally Sponsored Research The work described herein was supported in part by NIH grants 5P01AG12992, 5R01N834913A, 5P01NS31248, and NS29525-07. The Federal Government therefore may have certain rights in the invention.

### BACKGROUND OF THE INVENTION

The invention relates to genes involved in the onset of muscular dystrophy.

Muscular dystrophies constitute a heterogeneous group of disorders. Most are characterized by weakness and atrophy of the proximal muscles, although in rare myopathies such as “Miyoshi myopathy” symptoms may first arise in distal muscles. Of the various hereditary types of muscular dystrophy, several are caused by mutations or deletions in genes encoding individual components of the dystrophin-associated protein (DAP) complex. It is this DAP complex that links the cytoskeletal protein dystrophin to the extracellular matrix protein, laminin-2.

Muscular dystrophies may be classified according to the gene mutations that are associated with specific clinical syndromes. For example, mutations in the gene encoding the cytoskeletal protein dystrophin result in either Duchenne's Muscular Dystrophy or Becker's Muscular Dystrophy, whereas mutations in the gene encoding the extracellular matrix protein merosin produce Congenital Muscular Dystrophy. Muscular dystrophies with an autosomal recessive mode of inheritance include “Miyoshi myopathy” and the several limb-girdle muscular dystrophies (LGMD2). Of the limb-girdle muscular dystrophies, the deficiencies resulting in LGMD2C, D, E, and F result from mutations in genes encoding the membrane-associated sarcoglycan components of the DAP complex.

### SUMMARY OF THE INVENTION

A novel protein, designated dysferlin, is identified and characterized. The dysferlin gene is normally expressed in skeletal muscle cells and is selectively mutated in several families with the hereditary muscular dystrophies, e.g., Miyoshi myopathy (MM) and limb girdle muscular dystrophy-2B (LGMD2B). These characteristics of dysferlin render it a candidate disease gene for both MM and LGMD2B.

The present invention features an isolated DNA of 20-25 nucleotides, 30 nucleotides, 40 nucleotides, 50 nucleotides, 70 nucleotides, 100 nucleotides, 150 nucleotides, 200 nucleotides or 300 nucleotides in length. The isolated DNA includes a nucleotide sequence selected from the sequence of nucleotides 911-930 of SEQ ID NO: 1, 929-948 of SEQ ID NO: 1, 1019-1038 of SEQ ID NO: 1, 1392-1411 of SEQ ID NO: 1, 1424-1443 of SEQ ID NO: 1, 1484-1503 of SEQ ID NO: 1, 1499-1518 of SEQ ID NO: 1, 1543-1565 of SEQ ID NO: 1, 1715-1734 of SEQ ID NO: 1, 1740-1759 of SEQ ID NO: 1, 2241-2260 of SEQ ID NO: 1, 2864-2883 of SEQ ID NO: 1, 2978-2997 of SEQ ID NO: 1, 3057-3076 of SEQ ID NO: 1, 3198-3217 of SEQ ID NO: 1, 3252-3271 of SEQ ID NO: 1, 4356-4375 of SEQ ID NO: 1, 4665-4684 of SEQ ID NO: 1, 5015-5034 of SEQ ID NO: 1, 5610-5629 of SEQ ID NO: 1, 5726-5735 of SEQ ID NO: 1, 6035-6054 of SEQ ID NO: 1, 6179-6198 of SEQ ID NO: 1, 6243-6263 of SEQ ID NO: 1, and 6529-6548 of SEQ ID NO: 1. Each of these nucleotide sequences encompasses a mutation identified in a dysferlin gene.

Also within the invention is an isolated DNA which includes a nucleotide sequence of the sequences of nucleotides:

911-930 of SEQ ID NO: 1, wherein nucleotide C at 920 is T;

929-948 of SEQ ID NO: 1, wherein nucleotide C at 938 is G;

1019-1038 of SEQ ID NO: 1, wherein nucleotide G at 1028 is T;

1392-1411 of SEQ ID NO: 1, wherein nucleotide T at 1401 is C;

1424-1443 of SEQ ID NO: 1, wherein nucleotide A at 1433 is C;

1499-1518 of SEQ ID NO: 1, wherein nucleotide G at 1508 is T;

1715-1734 of SEQ ID NO: 1, wherein nucleotide A at 1724 is G;

1740-1759 of SEQ ID NO: 1, wherein nucleotide T at 1749 is C;

2241-2260 of SEQ ID NO: 1, wherein nucleotide T at 2250 is C;

2864-2883 of SEQ ID NO: 1, wherein nucleotide A at 2873 is G;

2978-2997 of SEQ ID NO: 1, wherein nucleotide G at 2987 is A;

3057-3076 of SEQ ID NO: 1, wherein nucleotide G at 3066 is A;

3198-3217 of SEQ ID NO: 1, wherein nucleotide G at 3207 is T;

3252-3271 of SEQ ID NO: 1, wherein nucleotide G at 3261 is T;

4356-4375 of SEQ ID NO: 1, wherein nucleotide G at 4365 is T;

5015-5034 of SEQ ID NO: 1, wherein nucleotide A at 5024 is G,

5610-5629 of SEQ ID NO: 1, wherein nucleotide G at 5619 is A;

5726-5735 of SEQ ID NO: 1, wherein nucleotide G at 5735 is T;

6035-6054 of SEQ ID NO: 1, wherein nucleotide G at 6044 is T;

6179-6198 of SEQ ID NO: 1, wherein nucleotide G at 6188 is T; and

6243-6263 of SEQ ID NO: 1, wherein nucleotide T at 6253 is deleted. Each of these nucleotide sequences contains a mutation found in a dysferlin gene and the wild type sequences flanking the mutation.

Also within the invention is an isolated DNA which includes a nucleotide sequence selected from GCAGGTGCGTGGGATGGACG (SEQ ID NO: 232) or CATATCCTCTTCATCCCTGC (SEQ ID NO: 233). Each of these nucleotide sequences contains a mutation which is an insertion of several nucleotides in a dysferlin gene.

The isolated DNA of the present invention can be either single-stranded or double-stranded nucleic acids.

Also within the invention is a pair of single stranded oligonucleotides which are different from each other, each selected from SEQ ID NOs: 234-283. These pairs of single stranded oligonucleotides can be used for exon amplification of the dysferlin gene.

Methods of identifying mutations in a dysferlin sequence are useful for predicting or diagnosing disorders associated with dysferlin, e.g., MM and LGMD2b. Thus, another aspect of the invention provides a method for identifying whether a patient, a fetus, or a pre-embryo is at risk for having a dysferlin-related disorder by providing a biological sample from the patient, fetus, or pre-embryo, and determining whether the sample contains a mutation in a dysferlin gene. The mutation can be:

nucleotide C at 920 of SEQ ID NO: 1 is T,

nucleotide C at 938 of SEQ ID NO: 1 is G,

nucleotide G at 1028 of SEQ ID NO: 1 is T.

nucleotide T at 1401 of SEQ ID NO: 1 is C,

nucleotide A at 1433 of SEQ ID NO: 1 is C,

nucleotide G at 1508 of SEQ ID NO: 1 is T,

nucleotide A at 1724 of SEQ ID NO: 1 is G,

nucleotide T at 1749 of SEQ ID NO: 1 is C,

nucleotide T at 2250 of SEQ ID NO: 1 is C,

nucleotide A at 2873 of SEQ ID NO: 1 is G,

nucleotide G at 2987 of SEQ ID NO: 1 is A,

nucleotide G at 3066 of SEQ ID NO: 1 is A,

nucleotide G at 3207 of SEQ ID NO: 1 is T,

nucleotide G at 3261 of SEQ ID NO: 1 is T,

nucleotide G at 4365 of SEQ ID NO: 1 is T,

nucleotide A at 5024 of SEQ ID NO: 1 is G,

nucleotide G at 5619 of SEQ ID NO: 1 is A,

nucleotide G at 5735 of SEQ ID NO: 1 is T,

nucleotide G at 6044 of SEQ ID NO: 1 is T,

nucleotide G at 6188 of SEQ ID NO: 1 is T,

nucleotide T at 6253 of SEQ ID NO: 1 is deleted,

an insertion of GTGCGTGG at 1553 of SEQ ID NO: 1, and

an insertion of ATCCTCTTCATC at 6538 of SEQ ID NO: 1.

In one embodiment of this method, the sample is incubated with a restriction enzyme and the presence or absence of a particular restriction site indicates the presence or absence or a particular mutation in the dysferlin gene. These methods can also be used to determine if a patient, fetus, or a pre-embryo is a carrier of a dysferlin mutation, for example in screening procedures. Other methods which can distinguish between different dysferlin alleles (e.g., a mutant allele and a normal allele) can be used to determine carrier status.

Another aspect of the invention provides a transgenic non-human mammal having a transgene which encodes a mutated dysferlin gene, wherein the mutated dysferlin gene

nucleotide C at 920 of SEQ ID NO: 1 is T,

nucleotide C at 938 of SEQ ID NO: 1 is G,

nucleotide G at 1028 of SEQ ID NO: 1 is T.

nucleotide T at 1401 of SEQ ID NO: 1 is C,

nucleotide A at 1433 of SEQ ID NO: 1 is C,

nucleotide G at 1508 of SEQ ID NO: 1 is T,

nucleotide A at 1724 of SEQ ID NO: 1 is G,

nucleotide T at 1749 of SEQ ID NO: 1 is C,

nucleotide T at 2250 of SEQ ID NO: 1 is C,

nucleotide A at 2873 of SEQ ID NO: 1 is G,

nucleotide G at 2987 of SEQ ID NO: 1 is A,

nucleotide G at 3066 of SEQ ID NO: 1 is A,

nucleotide G at 3207 of SEQ ID NO: 1 is T,

nucleotide G at 3261 of SEQ ID NO: 1 is T,

nucleotide G at 4365 of SEQ ID NO: 1 is T,

nucleotide A at 5024 of SEQ ID NO: 1 is G,

nucleotide G at 5619 of SEQ ID NO: 1 is A,

nucleotide G at 5735 of SEQ ID NO: 1 is T,

nucleotide G at 6044 of SEQ ID NO: 1 is T,

nucleotide G at 6188 of SEQ ID NO: 1 is T,

nucleotide T at 6253 of SEQ ID NO: 1 is deleted,

an insertion of GTGCGTGG at 1553 of SEQ ID NO: 1, and

an insertion of ATCCTCTTCATC at 6538 of SEQ ID NO: 1.

An “isolated DNA” is DNA which has a naturally occurring sequence corresponding to part or all of a given gene but is free of the two genes that normally flank the given gene in the genome of the organism in which the given gene naturally occurs. The term therefore includes a recombinant DNA incorporated into a vector, into an autonomously replicating plasmid or virus, or into the genomic DNA of a prokaryote or eukaryote. It also includes a separate molecule such as a cDNA, a genomic fragment, a fragment produced by polymerase chain reaction (PCR), or a restriction fragment, as well as a recombinant nucleotide sequence that is part of a hybrid gene, i.e., a gene encoding a fusion protein. The term excludes intact chromosomes and large genomic segments containing multiple genes contained in vectors or constructs such as cosmids, yeast artificial chromosomes (YACs), and P1-derived artificial chromosome (SAC) contigs.

A “noncoding sequence” is a sequence which corresponds to part or all of an intron of a gene, or to a sequence which is 5′ or 3′ to a coding sequence and so is not normally translated.

An expression control sequence is “operably linked” to a coding sequence when it is within the same nucleic acid and can effectively control expression of the coding sequence.

A “protein” or “polypeptide” is any chain of amino acids linked by peptide bonds, regardless of length or post-translational modification, e.g., glycosylation or phosphorylation.

As used herein, the term “percent sequence identity” means the percentage of identical subunits at corresponding positions in two sequences when the two sequences are aligned to maximize subunit matching, i.e., taking into account gaps and insertions. For purposes of the present invention, percent sequence identity between two polypeptides is to be determined using the Gap program and the default parameters as specified therein. The Gap program is part of the Sequence Analysis Software Package of the Genetics Computer Group, University of Wisconsin Biotechnology Center, 1710 University Avenue, Madison, Wis. 53705.

The algorithm of Myers and Miller, CABIOS (1989) can also be used to determine whether two sequences are similar or identical. Such an algorithm is incorporated into the ALIGN program (version 2.0) which is part of the GCG sequence alignment software package. When utilizing the ALIGN program for comparing amino acid sequences, a PAM120 weight residue table, a gap length penalty of 12, and a gap penalty of 4 can be used.

As used herein, the term “stringent hybridization conditions” means the following DNA hybridization and wash conditions: hybridization at 60° C. in the presence of 6 × SSC, 0.5% SDS, 5 × Denhardt's Reagent, and 100 μg/ml denatured salmon sperm DNA; followed by a first wash at room temperature for 20 minutes in 0.5 × SSC and 0.1% SDS and a second wash at 55° C. for 30 minutes in 0.2 × SSC and 0.1% SDS.

A “substantially pure protein” is a protein separated from components that naturally accompany it. The protein is considered to be substantially pure when it is at least 60%, by dry weight, free from the proteins and other naturally-occurring organic molecules with which it is naturally associated. Preferably, the purity of the preparation is at least 75%, more preferably at least 90%, and most preferably at least 99%, by weight. A substantially pure dysferlin protein can be obtained, for example, by extraction from a natural source, by expression of a recombinant nucleic acid encoding a dysferlin polypeptide, or by chemical synthesis. Purity can be measured by any appropriate method, e.g., column chromatography, polyacrylamide gel electrophoresis, or HPLC analysis. A chemically synthesized protein or a recombinant protein produced in a cell type other than the cell type in which it naturally occurs is, by definition, substantially free from components that naturally accompany it. Accordingly, substantially pure proteins include those having sequences derived from eukaryotic organisms but which have been recombinantly produced in E. coli or other prokaryotes.

An antibody that “specifically binds” to an antigen is an antibody that recognizes and binds to the antigen, e.g., a dysferlin polypeptide, but which does not substantially recognize and bind to other molecules in a sample (e.g., a biological sample) which naturally includes the antigen, e.g., a dysferlin polypeptide. An antibody that “specifically binds” to dysferlin is sufficient to detect a dysferlin polypeptide in a biological sample using one or more standard immunological techniques (for example, Western blotting or immunoprecipitation).

A “transgene” is any piece of DNA, other than an intact chromosome, which is inserted by artifice into a cell, and becomes part of the genome of the organism which develops from that cell. Such a transgene may include a gene which is partly or entirely heterologous (i.e., foreign) to the host organism, or may represent a gene homologous to an endogenous gene of the organism.

Unless otherwise defined, all technical and scientific terms used herein have the same meaning as commonly understood by one of ordinary skill in the art to which this invention belongs. Methods and materials similar or equivalent to those described herein can be used in the practice or testing of the present invention. The present materials, methods, and examples are illustrative only and not intended to be limiting. All publications, patent applications, patents, and other references mentioned herein are incorporated by reference in their entirety. In case of conflict, the present specification, including definitions, will control. All the sequences disclosed in the sequence listing are meant to be double-stranded except the sequences of oligonucleotides.

Other features and advantages of the invention will be apparent from the following detailed description, and from the claims.

### BRIEF DESCRIPTION OF THE DRAWINGS

FIG. 1A is a physical map of the MM locus. Arrows indicate the five new polymorphic markers and filled, vertical rectangular bores indicate the previously known polymorphic markers. The five ESTs that are expressed in skeletal muscle are highlighted in bold. Detailed information on the minimal tiling path of the PAC contig spanning the MM/LGMD2B region is provided in Liu et al., 1998, Genomics 49:23-29. The minimal candidate MM region is designated by the solid bracket (top) and compared to the previous candidate region (dashed bracket). TGFA and ADD2 are transforming growth factor alpha and β-adducin 2.

FIG. 1B is a representation of the dysferlin cDNA clones. The probes used in the three successive screens are shown in bold (130347, cDNA10, A27-F2R2). The two most 5′ cDNA clones are also shown (B22, B33). The 6.9 kb cDNA for dysferlin (SEQ ID NO: 1) is illustrated at the bottom with start and stop codons as shown.

FIG. 1C is a representation of the predicted dysferlin protein. The locations of four C2 domains (SEQ ID NOs: 86-89) are indicated by stippled boxes, while the putative transmembrane region is hatched. Vertical lines above the cDNA denote the positions of the mutations in Table 2; the associated labels indicate the phenotypes (MM—Miyoshi myopathy; LGMD—limb girdle muscular dystrophy; DMAT—distal myopathy with anterior tibial onset).

FIG. 2 is the sequence of the predicted 2,080 amino acids of dysferlin (SEQ ID NO: 2). The predicted membrane spanning residues are in bold at the carboxy terminus (residues 2047-2063). Partial C2 domains are underlined. Bold, underlined sequences are putative nuclear targeting residues. Possible membrane retention sequences are enclosed within a box.

FIG. 3 is a comparison of the Kyle-Doolittle hydrophobicity plots of the dysferlin protein and fer-1. On the Y-axis, increasing positivity corresponds to increasing hydrophobicity. Both proteins have a single, highly hydrophobic stretch at the carboxy terminal end (arrow). Both share regions of relative hydrophilicity approximately at residue 1,000 (arrowhead).

FIG. 4 is a SSCP analysis of a representative pedigree with dysferlin mutations. Each member of the pedigree is illustrated above the corresponding SSCP analysis. For each affected individual (solid symbols) shifts are evident in alleles 1 and 2, corresponding respectively to exons 36 and 54. As indicated, the allele 1 and 2 variants are transmitted respectively from the mother and the father. The two affected daughters in this pedigree have the limb girdle muscular dystrophy (LGMD) phenotype while their affected brother has a pattern of weakness suggestive of Miyoshi myopathy (MM).

FIG. 5 is a representation of the genomic structure of dysferlin. The 55 exons of the dysferlin gene and their corresponding SEQ ID NOs are indicated below the 6911 bp cDNA (solid line). The cDNA sequences corresponding to SEQ ID NO: 1 and SEQ ID NO: 3 are shown relative to the 6911 bp cDNA.

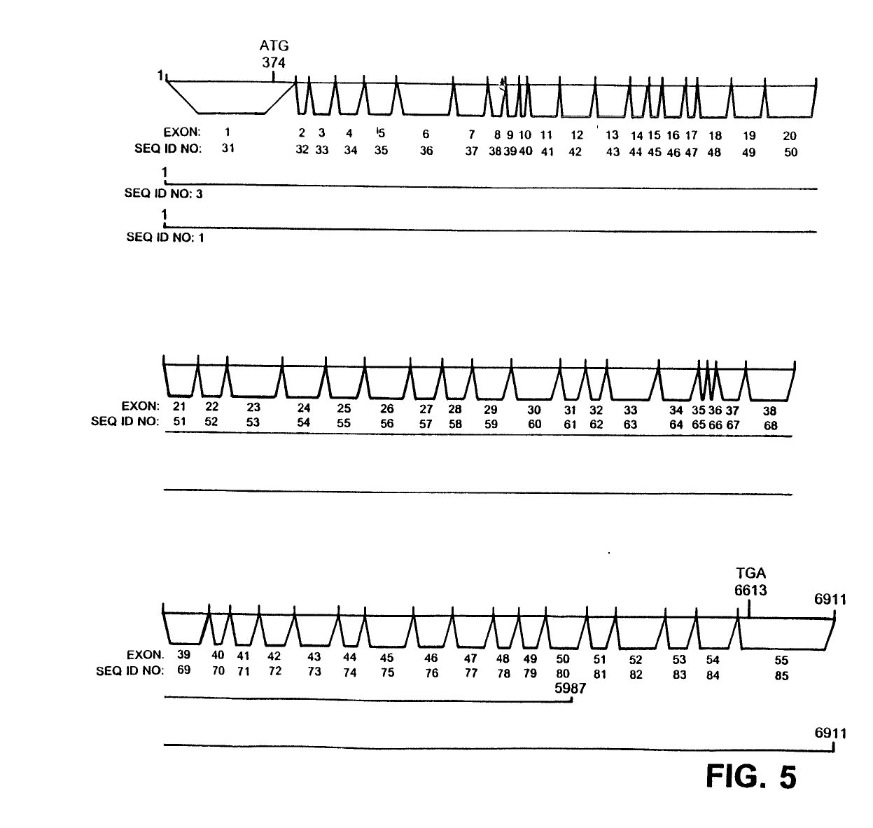

### DETAILED DESCRIPTION

The Miyoshi myopathy (MM) locus maps to human chromosome 2p12-14 between the genetic markers D2S292 and D2S286 (Bejaoui et al., 1995, Neurology 45:768-72). Further refined genetic mapping in MM families placed the MM locus between markers GGAA-P7430 and D2S2109 (Bejaoui et al., 1998, Neurogenetics 1:189-96). Independent investigation has localized the limb-girdle muscular dystrophy (LGMD-2B) to the same genetic interval (Bashir et al., 1994, Hum. Molec. Genetics 3:455-57; Bashir et al., 1996, Genomics 33:46-52; Passos-Bueno et al., 1995, Genomics 27:192-95). Furthermore, two large, inbred kindreds have been described whose members include both MM and LGMD2B patients (Weiler et al., 1996, Am. J. Hum. Genet. 59:872-78; Illarioshkin et al., 1997, Genomics 42:345-48). In these familial studies, the disease gene(s) for both MM and LGMD2B map to essentially to the same genetic interval. Moreover, in both pedigrees, individuals with MM or LGMD2B phenotypes share the same haplotypes. This raises the intriguing possibility that the two diseases may arise from the same gene defect and that a particular disease phenotype is the result of modification by additional factors.

A 3-Mb PAC contig spanning the entire MM/LGMD2B candidate region was recently constructed to facilitate the cloning of the MM/LGMD2E gene(s) (Liu et al., 1998, Genomics 49:23-29). This high resolution PAC contig resolved the discrepancies of the order of markers in previous studies (Bejaoui et al., 1998, Neurogenetics 1:189-96; Bashir et al., 1996, Genomics 33:46-52; Hudson et al., 1995, Science 270:1945-54). The physical size of the PAC contig also indicated that the previous minimal size estimation based on YAC mapping data was significantly underestimated.

Identification of Repeat Sequences and Repeat Typing

The PAC contig spanning the MM/LGMD2B region (Liu et al., 1998, Genomics 49:23-29) was used as a source for the isolation of new informative markers to narrow the genetic interval of the disease gene(s). DNA from the PAC clones spanning the MM/LGMD2B region was spotted onto Hybond N+™ membrane filters (Amersham, Arlington Heights, Ill.). The filters were hybridized independently with the following γ-³²P (Du Pont, Wilmington, Del.) labeled repeat sequences: (1) (CA)₁₅; (2) pool of (ATT)₁₀, (GATA)₈ and (GGAA)₈; (3) pool of (GAAT)₈, (GGAT)₈ and (GTAT)₈; and (4) pool of (AAG)₁₀ and (ATC)₁₀. Hybridization and washing of the filters were carried out at 55° C. following standard protocols (Sambrook et al., 1989, Molecular Cloning: A Laboratory Manual (2nd Edition), Cold Spring Harbor Press, N.Y.).

Miniprep DNAs of PAC clones containing repeat sequences were digested with restriction enzymes HindIII and PstI and ligated into pBluescript II (KS+) vector which is (Stratagene, La Jolla, Calif.) digested with the same enzymes. Filters of the PAC subclones were hybridized to the γ-³²P labeled repeats that detected the respective PACs. For clones with an insert size greater than 1 kb the repeat sequences of which could not be identified by a single round of sequencing, the inserts were further subcloned by digestion with HaeIII and ligation in EcoRV-digested pZero-2.1 vector (Invitrogen, Inc., Carlsbad, Calif.). Miniprep DNAs of the positive subclones were subjected to manual dideoxy sequencing with Sequenase™ enzyme (US Biochemicals, Inc., Cleveland, Ohio). Primer pairs for amplifying the repeat sequences were selected using the computer program Oligo (Version 4.0, National Biosciences, Inc., Plymouth, Minn.). Primer sequences are shown in Table 1.

TABLE 1
New Polymorphic Markers Mapped to the MM/LGMD2B Region

| | | | Annealing | Size in | No. of | |
|---|---|---|---|---|---|---|
| Marker | Repeat | Primers (5′ to 3′) | Tm (° C.) | PAC (bp) | alleles¹ | Het² |
| PAC3-H52 | CA | GATCTAACCCTGCTGCTCACC | 57 | 138 | 10 | 0.82 |
| | | (SEQ ID NO:120) | | | | |
| | | CTGGTGTGTTGCAGAGCGCTG | | | | |
| | | (SEQ ID NO:121) | | | | |
| Cy172-H32³ | CCAT | CCTCTCTTCTGCTGTCTTCAG | 56 | 199 | 7 | 0.72 |
| | | (SEQ ID NO:122) | | | | |
| | | TGTGTCTGGTTCCACCTTCGT | | | | |
| | | (SEQ ID NO:123) | | | | |
| PAC35-PH2 | CAT | TCCAAATAGAAATGCCTGAAC | 56 | 161 | 5 | 0.30 |
| | | (SEQ ID NO:124) | | | | |
| | | AGGTATCACCTCCAAGTGTTG | | | | |
| | | (SEQ ID NO:125) | | | | |
| PAC16-H41 | Complex | TACCAGCTTCAGAGCTCCCTG | 58 | 280 | 4 | 0.41 |
| | | (SEQ ID NO:126) | | | | |
| | | TTGATCAGGGTGCTCTTGG | | | | |
| | | (SEQ ID NO:127) | | | | |
| Cy7-PH3 | AAGG | GGAGAATTGCTTGAACCCAG | 56 | 211 | 4 | 0.32 |
| | | (SEQ ID NO:128) | | | | |
| | | TGGCTAATGATGTTGAACATTT | | | | |
| | | (SEQ ID NO:129) | | | | |

¹Observed in 50 unrelated caucasians.
²Heterozygosity index.
³Located within intron 2 of the dysferlin gene.
All oligonucleotides were synthesized by Integrated DNA Technologies, Inc. (Coralville, IA). PCR typing of the repeat markers followed previously described protocols (Bejaoui et al., 1995, Neurology 45:768-772).

Identification of Repeat Markers and Haplotype Analysis

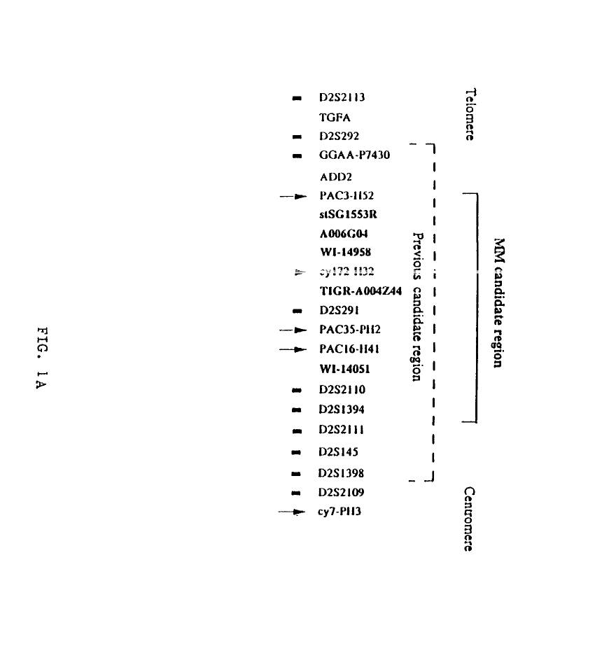

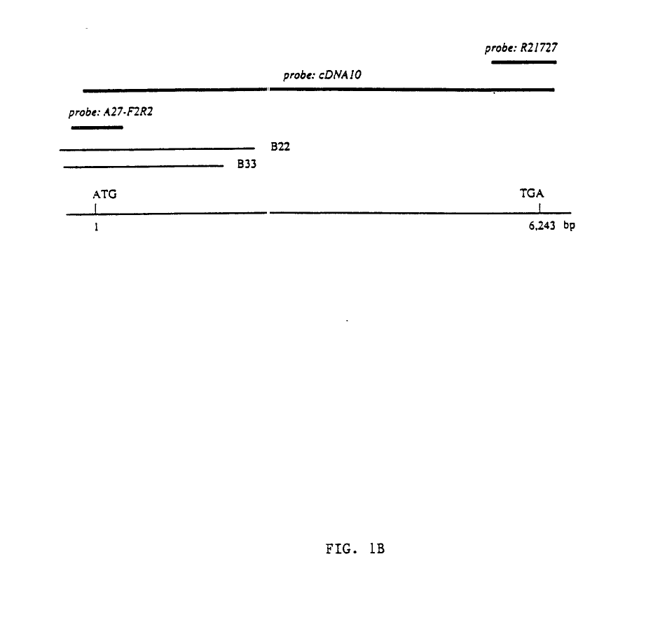

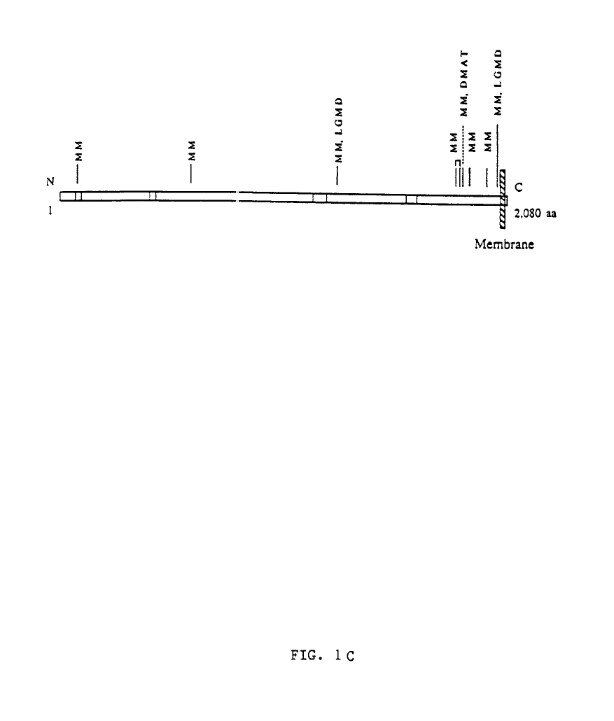

After hybridization with labeled repeat oligos, 17 different groups of overlapping PACs were identified that contained repeat sequences. Some groups contained previously identified repeat markers. For example, five groups of PACs were positively identified by a pool of repeat probes including (ATT)₁₀, (GATA) ₈, and (GGAA)₈. Of these, three groups contained known markers GGAA-P7430 (GGAA repeat), D2S1394 (GATA repeat) and D2S1398 (GGAA repeat) (Hudson et al., 1992, Nature 13:622-29; Gastier et al., 1995, Hum. Molecular Genetics 4:1829-36). No attempt was made to isolate new repeat markers from these PACs and they were not further analyzed. Similarly, seven groups of PACs that contained known CA repeat markers were excluded. Seven groups of PACs that contained unidentified repeats were retained for further analysis. For each group, the PAC containing the smallest insert was selected for subcloning. Subclones were re-screened and positive clones were sequenced to identify repeats. In total, seven new repeat sequences were identified within the MM/LGMD2B PAC contig. Of these, five are polymorphic within the population that was tested. The information for these five markers is summarized in Table 1. Based on the PAC contig constructed previously across the MM candidate locus (Liu et al., 1998, Genomics 48:23-29), the five new markers and ten previously published polymorphic markers were placed in an unambiguous order (FIG. 1).

These markers were analyzed in a large, consanguineous MM family (Bejaoui et al., 1995, Neurology 45: 768-72; Bejaoui et al., 1998, Neurogenetics 1:189-96). Because MM is a recessive condition, the locus can be defined by identifying legions of the genome that show homozygosity in affected individuals. Conversely, because of the high penetrance of this adult-onset condition, unaffected adult individuals are not expected to be homozygous by descent across the region. Analysis of haplotype homozygosity in this pedigree indicates that the disease gene lies between markers D2S2111 and PAC3-H52. Based on the PAC mapping data, the physical distance for this interval is approximately 2.0 Mb. No recombination events were detected between four informative markers (markers cy172-H32 to PAC16-H41) and the disease locus in family MM-21 (FIG. 1A).

Identification of Five Muscle-Expressed ESTs

Twenty-two ESTs and two genes (transforming growth factor alpha [TGFα] and beta-adducin [ADD2]) were previously mapped to the MM/LGMD2B PAC contig (FIG. 1A) (Liu et al., 1998, Genomics 48:23-29). Two μl (approximately 0.1 ng/μl) of Marathon-read skeletal muscle cDNA (Clontech, Palo Alto, Calif.) were used as template in a 10 μl PCR reaction for analysis of muscle expression of ESTs. The PCR conditions were the same as for the PCR typing of repeat markers. PCR analysis of skeletal muscle cDNA indicated that five of these ESTs (A006G04, stsG1553R, WI-14958, TIGR-A004Z44 and WI-14051) map within the minimal genetic MM interval of MM and are expressed in skeletal muscle.

Probes were selected corresponding to each of these five ESTs for Northern Blot analysis. cDNA clones (130347, 48106, 172575, 184080, and 510138) corresponding to the five ESTs that are expressed in muscle (respectively TIGR-A004Z44, WI-14051, WI-14958, stSG1553R and A006G04) were selected from the UniGene database (http:/www.ncbi.nlm.nih.gov/UniGene/) and obtained from Genome Systems, Inc. (St. Louis, Mo.). The cDNA probes were first used to screen the MM/LGMD2B PAC filters to confirm that they mapped to the expected position in the MM/LGMD2B contig.

A Northern blot (Clontech) of multiple human tissues was sequentially hybridized to the five cDNA probes and a control β-actin cDNA at 55° C. following standard hybridization and washing protocols (Sambrook et al., supra). Between hybridizations, probes were removed by boiling the blot at 95-100° C. for 4-10 min with 0.5% SDS. The blot was then re-exposed for 24 h to confirm the absence of previous hybridization signals before proceeding with the next round of hybridization.

The tissue distribution, intensity of the signals and size of transcripts detected by the five cDNA probes varied. Probes corresponding to ESTs stSG1553R, TIGR-A004Z44 and WI-14958 detected strong signals in skeletal muscle. In addition, the cDNA corresponding to TIGR-A004Z44 detected a 3.8 kb brain-specific transcript instead of the 8.5 kb message that was present in other tissues. It is likely that these five ESTs correspond to different genes since the corresponding cDNA probes used for Northern analysis derive from the 3′ end of messages, map to different positions in the MM/LGMD2B contig (FIG. 1A), and differ in their expression patterns.

Current database analysis suggests that three of these ESTs (stSG1553R, WI-14958 and WI-14051) do not match any known proteins (Schuler et al., 1996, Science 274:540-46). A006G04 has weak homology with a protein sequence of unknown function that derives from C. elegans. TIGR-A004Z44 has homology only to subdomains present within protein kinase C. Because the five genes corresponding to the ESTs are expressed in skeletal muscle and map within the minimal genetic interval of the MM/LGMD2B gene(s), they are candidate MM/LGMD2B gene(s).

Cloning of Dysferlin cDNA

EST TIGR-A004Z44 gave a particularly strong skeletal muscle signal on the Northern blot. Moreover, it is bracketed by genetic markers that show no recombination with the disease phenotype in family MM-21 (FIG. 1). The corresponding transcript was therefore cloned and analyzed as a candidate MM gene. From the Unigene database, a cDNA IMAGE clone (130347, 979 bp) was identified that contained the 483 bp EST TIGR-A004Z44.

Approximately 1×10⁶ recombinant clones of a λgt11 human skeletal muscle cDNA library (Clontech) were plated and screened following standard techniques (Sambrook et al., supra). The initial library screening was performed using the insert released from the clone 130347 that contains EST TIGR-A0044Z44, corresponding to the 3′ end of the gene. Positive phages were plaque purified and phage DNA was isolated according to standard procedures (Sambrook et al., supra). The inserts of the positive clones were released by EcoRI digestion of phage DNA and subsequently subcloned into the EcoRI site of pBluescript II (KS+) vector (Stratagene).

Fifty cDNA clones were identified when a human skeletal muscle cDNA library was screened with the 130347 cDNA. Clone cDNA10 with the largest insert (˜6.5 kb) (FIG. 1B) was digested independently with BamHI and PstI and further subcloned into pBluescript vector. Miniprep DNA of cDNA clones and subclones of cDNA10 was prepared using the Qiagen plasmid Miniprep kit (Valencia, Calif.). Sequencing was carried out from both ends of each clone using the SequiTherm EXCEL™ long-read DNA sequencing kit (Epicenter, Madison, Wisc.), fluorescent-labeled M13 forward and reverse primers, and a LI-COR sequencer (Lincoln, Nebr.). Assembly of cDNA contigs and sequence analysis were performed using Sequencher software (Gen;E Codes Corporation, Inc., Ann Arbor, Mich.).

Two additional screens, first with the insert of cDNA10 and then a 683 bp PCR product (A27-F2R2) amplified from the 5′ end of the cDNA contig, identified 87 additional cDNA clones. Clones B22 and B33 extended the 5′ end by 94 and 20 bp, respectively. The compiled sequence allowed for the generation of a sequence of 6.9 kb (SEQ ID NO: 1) (with 10-fold average coverage).

Although the 5′ end of the gene has not been further extended to the 8.5 kb predicted by Northern analysis, an open reading frame (ORF) of 6,243 bp has been identified within this 6.9 kb sequence. This ORF is preceded by an in-frame stop codon and begins with the sequence cgcaagcATGCTG (SEQ ID NO: 118); five of the first seven bp are consistent with the Kozak consensus sequence for a start codon (Kozak, 1989, Nucl. Acids Res. 15:8125-33; Kozak, 1989, J. Cell. Biol. 108:229-41). An alternate start codon, in the same frame, +75 bp downstream, appears less likely as a start site GAGACGATGGGG (SEQ ID NO: 119). Thus, the entire coding region of this candidate gene is believed to have been identified, as represented by the 6.9 kb sequence contig.

Identification of Mutations in Miyoshi Myopathy

Two strategies were used to determine whether this 6.9 kb cDNA (SEQ ID NO:1) is mutated in MM. First, the genomic organization of the corresponding gene was determined and the adjoining intronic sequence at each of the 55 exons which make up the cDNA was identified. To identify exon-intron boundaries within the gene, PAC DNA was extracted with the standard Qiagen-Mini Prep protocol. Direct sequencing was performed with DNA Sequence System (Promega, Madison, Wis.) using ³²P end-labeled primers (Benes et al., 1997, Biotechniques 23:98-100). Exon-intron boundaries were identified as the sites where genomic and cDNA sequences diverged. Second, in patients for whom muscle biopsies were available, RT-PCR was also used to prepare cDNA for the candidate gene from the muscle biopsy specimen.

Single strand conformational polymorphism analysis (SSCP) was used to screen each exon in patients from 12 MM families. Putative mutations identified in this way were confirmed by direct sequencing from genomic DNA using exon-specific intronic primers. Approximately 20 ng of total genomic DNA from immortalized lymphocyte cell lines were used as a template for PCR amplification analysis of each exon using primers (below) located in the adjacent introns. SSCP analysis was performed as previously described (Aoki et al., 1998, Ann. Neurol. 43:645-53). In patients for whom muscle biopsies were available, mRNA was isolated using RNA-STAT-60™ (Tel-Test, Friendswood, Tex.) and first-strand cDNA was synthesized from 1-2 μg total RNA with MMLV reverse transcriptase and random hexamer primers (Life Technologies, Gaithersburg, Md.). Three μl of this product were used for PCR amplification.

Eight sets of primers were designed for muscle cDNA, and overlapping cDNA fragments suitable for SSCP analysis were amplified. After initial denaturation at 94° C. for 2 min, amplification was performed using 30 cycles at 94° C. for 30 s, 56° C. for 30 s, and 72° C. for 60 s. The sequences of polymorphisms detected by SSCP analysis were determined by the dideoxy termination method using the Sequenase kit (US Biochemicals). In some instances, the base pair changes predicted corresponding changes in restriction enzyme recognition sites. Such alterations in restriction sites were verified by digesting the relevant PCR products with the appropriate restriction enzymes.

Primer pairs used for SSCP screening and exon sequencing are as follows:

(1) exon 3, F3261 5′-tctcttctcctagagggccatag-3′ (SEQ ID NO: 101) and R326 5′-ctgttcctccccatcgtctcatgg-3′ (SEQ ID NO: 102);

(2) exon 20, F3121 5′-gctcctcccgtgaccctctg-3′ (SEQ ID NO: 103) and R3121 5′-gggtcccagccaggagcactg-3′ (SEQ ID NO: 104);

(3) exon 36, F2102 5′-cccctctcaccatctcctgatgtg-3′ (SEQ ID NO: 105) and R2111 5′-tggcttcaccttccctctacctcgg-3′ (SEQ ID NO: 106);

(4) exon 49, F1081 5′-tcctttggtaggaaatctaggtgg-3′ (SEQ ID NO: 107) and R1081 5′-ggaagctggacaggcaagagg-3′ (SEQ ID NO: 108);

(5) exon 50, F1091 5′-atatactgtgttggaaatcttaatgag-3′ (SEQ ID NO: 109) and R1091 5′-gctggcaccacagggaatcgg-3′ (SEQ ID NO: 110);

(6) exon 51, F1101 5′-ctttgcttccttgcatccttctctg-3′ (SEQ ID NO: 111) and R1101 5′-agcccccatgtgcagaatggg-3′ (SEQ ID NO: 112);

(7) exon 52, F1111 5′-ggcagtgatcgagaaacccgg-3′ (SEQ ID NO: 113) and R1111 5′-catgccctccactggggctgg-3′ (SEQ ID NO: 114);

(8) exon 54, F1141 5′-ggatgcccagttgactccggg-3′ (SEQ ID NO: 115) and R1141 5′-ccccaccacagtgtcgtcagg-3′ (SEQ ID NO: 116);

(9) exon 29, F3031 5′-aagtgccaagcaatgagtgaccgg-3′ (SEQ ID NO: 184) and R3021 5′-ctcactcccacccaccacctg-3′ (SEQ ID NO: 185);

(10) exon 31, F2141 5′-gaatctgccataaccagcttcgtg-3′ (SEQ ID NO: 188) and R2141 5′-tatcaccccatagaggcctcgaag-3′ (SEQ ID NO: 189);

(11) exon 32, F2981 5′-cagccactcactctggcacctctg-3′ (SEQ ID NO: 190) and R2981 5′-agcccacagtctctgactctcctg-3′ (SEQ ID NO: 191);

(12) exon 43, F2031 5′-cagccaaaccatatcaacaatg-3′ (SEQ ID NO: 210) and R2021 5′-ctggggaggtgagggctctag-3′ (SEQ ID NO: 211);

(13) exon 44, F2011 5′-gaagtgttttgtctcctcctc-3′ (SEQ ID NO: 212) and R2011 5′-gcaggcagccagcccccatc-3′ (SEQ ID NO: 213);

(14) exon 46, F1041 5′-ctcgtctatgtcttgtgcttgctc-3′ (SEQ ID NO: 216) and R1051 5′-caccatggtttggggtcatgtgg-3′ (SEQ ID NO: 217).

These primers were used in SSCP screening and exon sequencing, and identified eighteen different mutations in fifteen families (Table 2).

TABLE 2
Mutations in Dysferlin in Distal Myopathy and LGMD¹

| | | | | | | | Change of |
|---|---|---|---|---|---|---|---|
| | Nucleotide | | | | | | restriction |
| Name | Change | Exon | Consequence | Origin | Family Name | Allele | site |
| Mutations | | | | | | | |
| 537insA | ins of A at | 3 | Frameshift | Arabic | MM59 | Hom | no change |
| | 537 | | | | | | |
| Q605X | CAG to TAG at | 20 | Stop at 605 | Grench | MM67 | Hom | −Pst I, |
| | 2186 | | | | | | −Fnu 4H I¹ |
| I1298V | ATC to GTC at | 36 | Amino acid | Italian | MM, LGMD56 | Het | −BamHI, |
| | 4265 | | change | | | | −BStYI; |
| | | | | | | | +Ava II |
| E1883X | GAG to TAG at | 49 | Stop at 1883 | English | MM8 | Het | no change |
| | 5870 | | | | | | |
| H1857R | CAT to CGT at | 50 | Amino acid | English | MM50 | Het | no change |
| | 5943 | | change | | | | |
| 5966delG | del of G at | 50 | Frameshift | Spanish | DMAT71 | Hom | no change |
| | 5966 | | | | | | |
| 5966delG | del of G at | 50 | Frameshift | Spanish | MM75 | Hom | no change |
| | 5966 | | | | | | |
| 6071/6072delAG | del of AG at | 51 | Frameshift | English | MM58 | Het | no change |
| | 6071/6072 | | | | | | |
| 6319 +1G to A | Ggt to Gat at | 52 | 5′ splice site | English | MM8 | Het | no change |
| | 6319 + 1 | | | | | | |
| R2042C | CGT to TGT at | 54 | Amino acid | Italian | MM56 | Het | −Fnu4HI |
| | 6497 | | change | | | | |
| R1046H | CGC to CAG at | 29 | Amino acid | Japanese | MM10 | Hom | −HinPI, |
| | 3510 | | change | | | | −Fsp I |
| 3746delG | del of G at | 31 | Frameshift | Japanese | MM17 | Hom | −MboII |
| | 3746 | | | | | | |

| | | | | | | | |
|---|---|---|---|---|---|---|---|
| Q1160X | CAG to TAG at | 32 | Stop at 1160 | Mexican | MM46 | Hom | −ScrFI, |
| | 3851 | | | | | | −BstNI, |
| | | | | | | | +MaeI, |
| | | | | | | | +BfaI |
| 5122/5123delCA | del of CA at | 43 | Frameshift | Japanese | MM14 | Het | no change |
| | 5122/5123, A | | | | | | |
| | to T at 5121 | | | | | | |
| R1586X | CGA to TGA at | 43 | Stop at 1586 | Japanese | MM12 | Hom | +Dde I |
| | 5129 | | | | | | |
| 5245delG | del of G at | 44 | Frameshift | French | MM63 | Hom | −Bpm I, |
| | 5245 and G to | | | | | | −BanII |
| | C at 5249, or | | | | | | +AvaII, |
| | G to C at | | | | | | +Sau96I |
| | 5245 and del | | | | | | |
| | G at 5249 | | | | | | |
| E1732X | GAG to TAG at | 46 | Stop at 1732 | Spanish | MM73 | Het | +Mbo II |
| | 5567 | | | | | | |

¹MM: Miyoshi myopathy; DMAT: distal myopathy with anterior tibial onset; LGMD: limb girdle muscular dystrophy
²+: create a new restriction site, −: eliminate an existing restriction site.

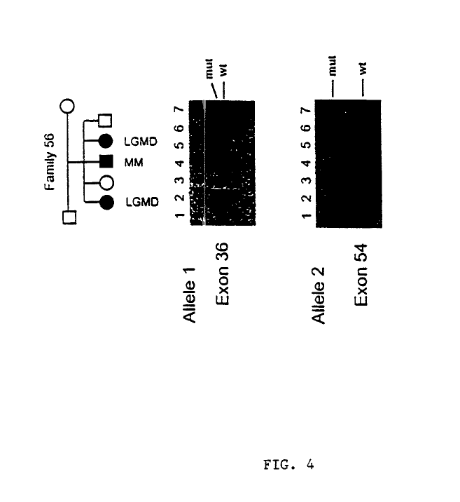

Twelve of the eighteen mutations block dysferlin expression, either through nonsense or frameshift changes; Seven of these twelve are homozygous, and are thus expected to result in complete loss of dysferlin function. For each mutated exon in these patients, at least 50 control DNA samples (100 chromosomes) were screened to determine the frequencies of the sequence variants. When possible, the parents and siblings of affected individuals were also screened to verify that defined mutations were appropriately co-inherited with the disease in each pedigree (FIG. 4). In two families (50, 58 in Table 2) heterozygous mutations were identified in one allele (respectively a missense mutation and a 2 bp deletion). Mutations in the other allele are presumed to have not been detected (or in three of the screened MM families) either because the mutant and normal SSCP products are indistinguishable or because the mutation lies outside of a coding sequence (i.e., in the promoter or a regulatory region of an intron). The disease-associated mutations did not appear to arise in the population as common polymorphisms.

More mutations can be identified by using appropriate primer pairs to amplify an exon and analyze its sequence. The following primer pairs are useful for exon amplification.

| Exon | Code | Primer Sequence |
|---|---|---|
| 1 | F408 | 5′-gacccacaagcggcgcctcgg-3′ {SEQ ID NO: 130} |
| | F4101 | 5′-gaccccggcgagggtggtcgg-3′ {SEQ ID NO: 131} |
| 2 | F4111 | 5′-tgtctctccattctcccttttgtg-3′ {SEQ ID NO: 132} |
| | R4111 | 5′-aggacactgctgagaaggcacctc-3′ {SEQ ID NO: 133} |
| 3 | F3262 | 5-agtgccctggtggcacgaagg-3′ {SEQ ID NO: 134} |
| | R3261 | 5-cctacctgcaccttcaagccatgg-3′ {SEQ ID NO: 135} |
| 4 | F3251 | 5-cagaagagccagggtgccttagg-3′ {SEQ ID NO: 136} |
| | R3251 | 5-ccttggaccttaacctggcagagg-3′ {SEQ ID NO: 137} |
| 5 | F3242 | 5-cgaggccagcgcaccaacctg-3′ {SEQ ID NO: 138} |
| | R3242 | 5-actgccggccattcttgctggg-3′ {SEQ ID NO: 139} |
| 6 | F3231 | 5-ccaggcctcattagggccctc-3′ {SEQ ID NO: 140} |
| | R3231 | 5-ctgaagaggagcctggggtcag-3′ {SEQ ID NO: 141} |
| 7 | F3222 | 5-ctgagatttctgactcttggggtg-3′ {SEQ ID NO: 142} |
| | R3211 | 5-aaggttctgccctcatgccccatg-3′ {SEQ ID NO: 143} |
| 8 | F3561 | 5-ctggcctgagggatcagcagg-3′ {SEQ ID NO: 144} |
| | R3561 | 5-gtgcatacatacagcccacggag-3′ {SEQ ID NO: 145} |
| 9 | F3551 | 5-gagctattgggttggccgtgtggg-3′ {SEQ ID NO: 146} |
| | R3552 | 5-accaacacggagaagtgagaactg-3′ {SEQ ID NO: 147} |
| 10 | F3201 | 5-ccacactttatttaacgctttggcgg-3′ {SEQ ID NO: 148} |
| | R3201 | 5-cagaaccaaaatgcaaggatacgg-3′ {SEQ ID NO: 149} |
| 11 | F3191 | 5-cttctgattctgggatcaccaaagg-3′ {SEQ ID NO: 150} |
| | F3191 | 5-ggaccgtaaggaagacccaggg-3′ {SEQ ID NO: 151} |
| 12 | F3181 | 5-cctgtgctcaggagcgcatgaagg-3′ {SEQ ID NO: 152} |
| | R3181 | 5-gcagacctcccacccaagggcg-3′ {SEQ ID NO: 153} |
| 13 | F3171 | 5-gagacagatgggggacagtcaggg-3′ {SEQ ID NO: 154} |
| | R3171 | 5-cctcccgagagaaccctcctg-3′ {SEQ ID NO: 155} |
| 14 | F3161 | 5-gggagcccagagtccccatgg-3′ {SEQ ID NO: 156} |
| | R3161 | 5-gggcctccttgggtttgctgg-3′ {SEQ ID NO: 157} |
| 15 | F3541 | 5-gcctccccagcatcctgccgg-3′ {SEQ ID NO: 158} |
| | R3541 | 5-tcactgagccgaatgaaactgagg-3′ {SEQ ID NO: 159} |
| 16 | F3531 | 5-tgtggcctgagttcctttcctgtg-3′ {SEQ ID NO: 160} |
| | R3531 | 5-ggtcaaagggcagaacgaagaggg-3′ {SEQ ID NO: 161} |
| 17 | F3151 | 5-cccgtccttctcccagccatg-3′ {SEQ ID NO: 162} |
| | R3151 | 5-ctcccccggttgtccccaagg-3′ {SEQ ID NO: 163} |
| 18 | F3141 | 5-cgacccctctgattgccacttgtg-3′ {SEQ ID NO: 164} |
| | R3141 | 5-ggcatcctgcccttgccaggg-3′ {SEQ ID NO: 165} |
| 19 | F3522 | 5-tctgtcccccctgctccttg-3′ {SEQ ID NO: 166} |
| | R3522 | 5-cttccctgccccgacgcccag-3′ {SEQ ID NO: 167} |
| 20 | F3121 | 5-gctcctcccgtgaccctctgg-3′ {SEQ ID NO: 103} |
| | R3121 | 5-gggtcccagccaggagcactg-3′ {SEQ ID NO: 104} |
| 21 | F3111 | 5-cagcgctcaggcccgtctctc-3′ {SEQ ID NO: 168} |
| | R3111 | 5-tgcataggcatgtgcagctttggg-3′ {SEQ ID NO: 169} |
| 22 | F3512 | 5-catgcaccctctgccctgtgg-3′ {SEQ ID NO: 170} |
| | R3512 | 5-agttgagccaggagaggtggg-3′ {SEQ ID NO: 171} |
| 23 | F3101 | 5-catcaggcgcattccatctgtccg-3′ {SEQ ID NO: 172} |
| | R3091 | 5-agcaggagagcagaagaagaaagg-3′ {SEQ ID NO: 173} |
| 24 | F3082 | 5-gtgtgtcaccatccccaccccg-3′ {SEQ ID NO: 174} |
| | R3082 | 5-caagagatgggagaaaggccttatg-3′ {SEQ ID NO: 175} |
| 25 | F3073 | 5-ctgggacatccggatcctgaagg-3′ {SEQ ID NO: 176} |
| | R3073 | 5-tccaggtagtgggaggcagagg-3′ {SEQ ID NO: 177} |
| 26 | F3061 | 5-tcccactacctggagctgccttgg-3′ {SEQ ID NO: 178} |
| | R3051 | 5-ggctctccccagccctccctg-3′ {SEQ ID NO: 179} |
| 27 | F3601 | 5-cagagcagcagagactctgaccag-3′ {SEQ ID NO: 180} |
| | R3601 | 5-tagaccccacctgcccctgag-3′ {SEQ ID NO: 181} |
| 28 | F3501 | 5-tcctctcattgcttgcctgttcgg-3′ {SEQ ID NO: 182} |
| | R3501 | 5-ttgagagcttgccggggatgg-3′ {SEQ ID NO: 183} |
| 29 | F3031 | 5-aagtgccaagcaatgagtgaccgg-3′ {SEQ ID NO: 184} |
| | R3021 | 5-ctcactcccacccaccacctg-3′ {SEQ ID NO: 185} |
| 30 | F3011 | 5-cccaccggcctctgagtctgc-3′ {SEQ ID NO: 186} |
| | R3001 | 5-accctacccaagccaggacaagtg-3′ {SEQ ID NO: 187} |
| 31 | F2141 | 5-gaatctgccataaccagcttcgtg-3′ {SEQ ID NO: 188} |
| | R2141 | 5-tatcaccccatagaggcctcgaag-3′ {SEQ ID NO: 189} |
| 32 | F2981 | 5-cagccactcactctggcacctctg-3′ {SEQ ID NO: 190} |
| | R2981 | 5-agcccacagtctctgactctcctg-3′ {SEQ ID NO: 191} |
| 33 | F2131 | 5-acatctctcagggtccctgctgtg-3′ {SEQ ID NO: 192} |
| | R2211 | 5-cctgtgaggggacgaggcagg-3′ {SEQ ID NO: 193} |
| 34 | F2202 | 5-gccctgggtaagggatgctgattc-3′ {SEQ ID NO: 194} |
| | R2202 | 5-cctgcctgggcctcctggatc-3′ {SEQ ID NO: 195} |
| 35 | F2111 | 5-gagggtgatgggggccttagg-3′ {SEQ ID NO: 196} |
| | R2112 | 5-gcaatcagtttgaagaaggaaagg-3′ {SEQ ID NO: 197} |
| 36 | F2102 | 5-cccctctcaccatctcctgatgtg-3′ {SEQ ID NO: 105} |
| | R2111 | 5-ggcttcaccttccctctacctcgg-3′ {SEQ ID NO: 106} |
| 37 | F2101 | 5-cacctttgtctccattctacctgc-3′ {SEQ ID NO: 198} |
| | R2101 | 5-ctcccagcccccacgcccagg-3′ {SEQ ID NO: 199} |
| 38 | F2091 | 5-ctgagccactctcctcattctgtg-3′ {SEQ ID NO: 200} |
| | R2091 | 5-tggaaggggacagtagggagg-3′ {SEQ ID NO: 201} |
| 39 | F2081 | 5-ggccagtgcgttcttcctcctc-3′ {SEQ ID NO: 202} |
| | R2071 | 5-tccctgacctgcccatcatctc-3′ {SEQ ID NO: 203} |
| 40 | F2061 | 5-gcccctgtcaggcctggatgg-3′ {SEQ ID NO: 204} |
| | R2061 | 5-tgacccaggcctccctggagg-3′ {SEQ ID NO: 205} |
| 41 | F2051 | 5-ctgaaatggtctctttctttctac-3′ {SEQ ID NO: 206} |
| | R2051 | 5-cacaccgactgtcagactgaagag-3′ {SEQ ID NO: 207} |
| 42 | F2041 | 5-ttgtcccctcctctaatccccatg-3′ {SEQ ID NO: 208} |
| | R2041 | 5-gggttagggacgtcttcgagg-3′ {SEQ ID NO: 209} |
| 43 | F2031 | 5-cagccaaaccatatcaacaatg-3′ {SEQ ID NO: 210} |
| | R2021 | 5-ctggggaggtgagggctctag-3′ {SEQ ID NO: 211} |
| 44 | F2011 | 5-gaagtgttttgtctcctcctc-3′ {SEQ ID NO: 212} |
| | R2011 | 5-gcaggcagccagcccccatc-3′ {SEQ ID NO: 213} |
| 45 | F1021 | 5-gggtgcoctgtgttggctgac-3′ {SEQ ID NO: 214} |
| | R1031 | 5-gcaggcagccagcccccatc-3′ {SEQ ID NO: 215} |
| 46 | F1041 | 5-ctcgtctatgtcttgtgcttgctc-3′ {SEQ ID NO: 216} |
| | R1051 | 5-caccatggtttggggtcatgtgg-3′ {SEQ ID NO: 217} |
| 47 | F1061 | 5-tctcgcttccccagctcctgc-3′ {SEQ ID NO: 218} |
| | R1061 | 5-tctggagttcgaggactctggg-3′ {SEQ ID NO: 219} |
| 48 | F1071 | 5-agaagggtggggagagaacgg-3′ {SEQ ID NO: 220} |
| | R1071 | 5-cagctcagagcctgtggctgg-3′ {SEQ ID NO: 221} |
| 49 | F1082 | 5-aaggccttcccatcctttggtagg-3′ {SEQ ID NO: 222} |
| | R1082 | 5-acaacccagagggagcacggg-3′ {SEQ ID NO: 223} |
| 50 | F1092 | 5-gttgacgatgtatatactgtgttgg-3′ {SEQ ID NO: 224} |
| | R1091 | 5-gctggcaccacagggaatcgg-3′ {SEQ ID NO: 110} |
| 51 | F1102 | 5-gcctctctctaactttgcttccttg-3′ {SEQ ID NO: 225} |
| | R1101 | 5-agcccccatgtgcagaatggg-3′ {SEQ ID NO: 112} |
| 52 | F1112 | 5-ggctacaggctggcagtgatcgag-3′ {SEQ ID NO: 226} |
| | R1112 | 5-ttcccccatgccctccactgg-3′ {SEQ ID NO: 227} |
| 53 | F1121 | 5-agccttcgtgcccctaaccaagtg-3′ {SEQ ID NO: 228} |
| | R1121 | 5-ctgtgggcattggggctcagg-3′ {SEQ ID NO: 229} |
| 54 | F1141 | 5-ggatgcccagttgactccggg-3′ {SEQ ID NO: 115} |
| | R1141 | 5-ccccaccacagtgtcgtcagg-3′ {SEQ ID NO: 116} |
| 55 | F1151 | 5-gccccagtgggatcaccatg-3′ {SEQ ID NO: 230} |
| | R116 | 5-atgctggaggggaccccacgg-3′ {SEQ ID NO: 231} |

More mutations can also be identified by using appropriate exonic primer pairs to amplify an exon directly from a cDNA sample and analyze the exon sequence. The following exonic primer pairs are useful for exon amplification and mutation screening.

| Exon | Primer Sequence |
|---|---|
| 1 | GCCCAGCCAGGTGCAAAATG (SEQ ID NO: 234), |
| | CAAAGAGGGCTCGGAAAGGT (SEQ ID NO: 235), |
| 2 | ACCCACAAGCGGCGCCTCGG (SEQ ID NO: 236), |
| | TCGGAGTGGGACCTTGGCTT (SEQ ID NO: 237), |
| 3 | GCTCTGAGCTTCATGTGGTGGTC (SEQ ID NO: 238), |
| | CCAGGAATGGCTCCGCCTCATC (SEQ ID NO: 239), |
| 4 | CTCTGCCTGACCTGGATGTAGT (SEQ ID NO: 240), |
| | AGTCTCATTGAAGAGTGGGCTG (SEQ ID NO: 241), |
| 5 | CATCAAGCCTGTGGTCAAGGTTA (SEQ ID NO: 242), |
| | CTTTTCTCTCCAGAGGCGCTTCG (SEQ ID NO: 243), |
| 6 | TGCTGGGGCCAGAGGCTA (SEQ ID NO: 244), |
| | GTTCTGGTTCCACTGAGGG (SEQ ID NO: 245), |
| 7 | GTGGAGGTCAGCTTTGC (SEQ ID NO: 246), |
| | CTGGGAAGCCTGTGAACT (SEQ ID NO: 247), |
| 8 | GCTTCCTCCCCACTTTTGG (SEQ ID NO: 248), |
| | CGGCAGGCAGGTCATGTCG (SEQ ID NO: 249), |
| 9 | GAGGTCAGCATCGGGAACTAC (SEQ ID NO: 250), |
| | CTGGCTGCAGCCTGCGATGAG (SEQ ID NO: 251), |
| 10 | CTCCACGGAGGACGTGGACTC (SEQ ID NO: 252), |
| | CACCCGCTGGTATGCCACACGC (SEQ ID NO: 253), |
| 11 | CAGAACAGCCTGCCGGACAT (SEQ ID NO: 254), |
| | CTTGGGGTAGGTGAGGCCCG (SEQ ID NO: 255), |
| 12 | CGAGACTAAGTTGGCCCTTG (SEQ ID NO: 256), |
| | AGGTCTGTGGACCATTCCTCA (SEQ ID NO: 257), |
| 13 | CTTCCCAAGGATGACATTGAG (SEQ ID NO: 258), |
| | TCTCCAGTGGCTCCATGCGA (SEQ ID NO: 259), |
| 14 | CTCGAGTACCGCAAGACAG (SEQ ID NO: 260), |
| | CCCAGGTGGGGTTAAGGGTG (SEQ ID NO: 261), |
| 15 | TGGACAAGGACTCTTTTTC (SEQ ID NO: 262), |
| | CCTCAAAACCAGGAATATG (SEQ ID NO: 263), |
| 16 | GAGCTCATCCAGAGAGAGAAGC (SEQ ID NO: 264), |
| | CACTTCCATGAAGAGGGTGC (SEQ ID NO: 265), |
| 17 | GTGCAGTCCTGTGTCATCAG (SEQ ID NO: 266), |
| | GCTCCACCAATCGATGAAC (SEQ ID NO: 267), |
| 18 | GAGAAGACGTGCTCATCGAC (SEQ ID NO: 268), |
| | CAGCTGGTGGAACTGTCTTG (SEQ ID NO: 269), |
| 19 | GAAGATCCATCTGTGATTGGTG (SEQ ID NO: 270), |
| | CCTTGGAGAGGAGGTCATAGTC (SEQ ID NO: 271), |
| 20 | GATGTTCGAGCTGACCTGCAC (SEQ ID NO: 272), |
| | GTGTGGGTTTGGGATCCTGC (SEQ ID NO: 273), |
| 21 | GGCACCTGTGTACCGGACAG (SEQ ID NO: 274), |
| | GGATCACATCTCTGGTATT (SEQ ID NO: 275), |
| 22 | GTGGGTCGACCTATTTCCG (SEQ ID NO: 276), |
| | CAGCCTCCAGAAGGCATCC (SEQ ID NO: 277), |
| 23 | GAGGTTCATTTTCCCCTTCGAC (SEQ ID NO: 278), |
| | CAGTATTTTCTTCTCACCCTC (SEQ ID NO: 279), |
| 24 | GTCCCTTTTTGAGCAGAAAAC (SEQ ID NO: 280), |
| | CTTCATGGCAGCATAGTTCG (SEQ ID NO: 281), |
| 25 | CTTCATCCTGCTGCTGTTCC (SEQ ID NO: 282), |
| | GTGGTTCCAACTGTTTTATAC (SEQ ID NO: 283), |

The following mutations were identified by using the exonic primer pairs listed above.

| Nucleotide Position | | Restriction |
|---|---|---|
| (in SEQ ID NO:1) | Mutation | Site |
| 920 | C to T | −BanII, |
| | | −Bsp1286I |
| 938 | C to G | +RsaI |
| 1028 | G to T | no change |
| 1401 | T to C | +HhaI |
| 1433 | A to C | no change |
| 1493 | G to C | +HaeIII |
| 1508 | G to T | −Bsp1286 |
| | | −BanII |
| | | −NlaIV |
| 1553 | aberrant splicing, | no change |
| | insertion of 9 bp | |
| | of intronic sequence: | |
| | GTGCGTGGG | |
| 1724 | A to G | −NlaIII |
| 1749 | T to C | no change |
| 2250 | T to C | +BcgI |
| | | −NlaIII |
| 2873 | A to G | no change |
| 2987 | G to A | no change |
| 3066 | G to A | +AvaII |
| | | −HaeIII |
| 3207 | G to T | −BstEII |
| 3261 | G to T | +PleI |
| | | −HaeI |
| 4365 | G to T | +Alu |
| | | −RsaI |
| 4674 | C to T | no change |
| 5024 | A to G | −ApoI |
| | | −Tsp509I |
| 5619 | G to A | no change |
| 5735 | G to T | −SalI |
| | | −HincII |
| | | −TaqI |
| 6044 | G to T | +HinfI |
| | | −TfiI |
| | | −SfaNI |
| | | −FokI |
| 6253 | delete T | no change |
| 6188 | G to T | no change |
| 6253 | delete T | |
| 6538 | duplication of | |
| | 12 exonic bp: | |

Comparison of Dysferlin With Other Proteins

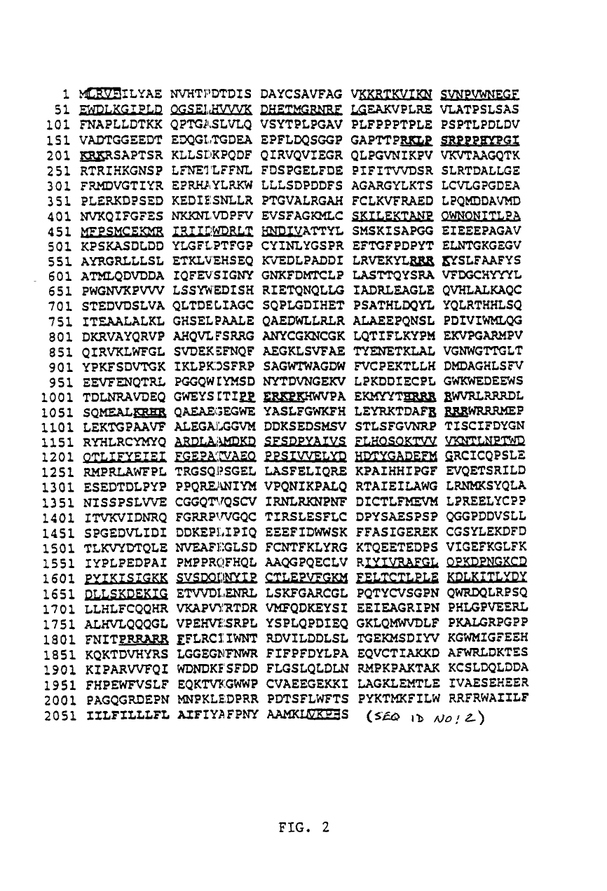

The 6,243 bp ORF of this candidate MM gene is predicted to encode 2,080 amino acids (FIGS. 1C and 2; SEQ ID NO: 2). At the amino acid level, this protein is highly homologous to the nematode (Caenorhabditis elegans) protein fer-1 (27% identical, 57% identical or similar: the sequence alignment and comparison was performed using http://vega.igh.cnrs.fr/bin/nph-alignquery.p1.) (Argon & Ward, 1980, Genetics 96:413-33; Achanzar & Ward, 1997, J. Cell Science 110:1073-81). This dystrophy-associated, fer-1-like protein has therefore been designated “dysferlin.”

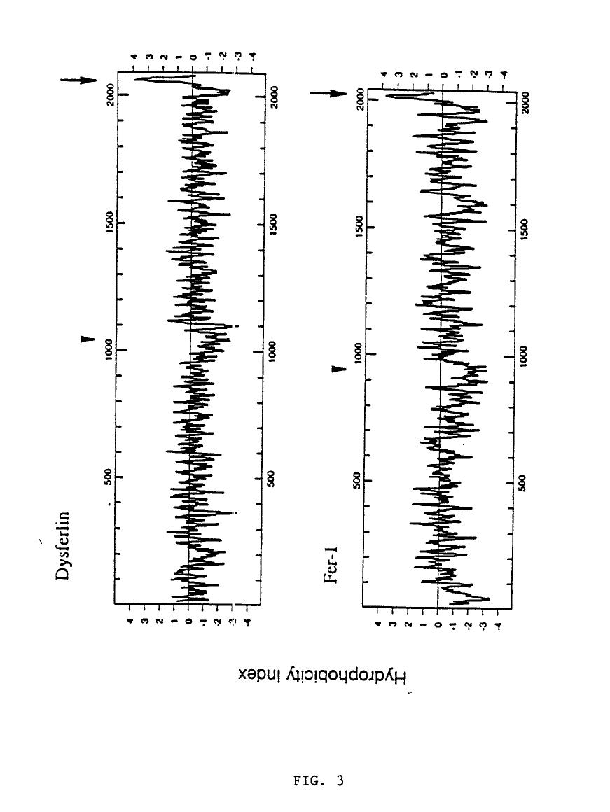

The fer-1 protein was originally identified through molecular genetic analysis of a class of fertilization-defective C. elegans mutants in which spermatogenesis is abnormal (Argon & Ward, 1980, Genetics 96:413-33). The mutant fer-1 spermatozoa have defective mobility and show imperfect fusion of membranous organelles (Ward et al., 1981, J. Cell Bio. 91:26-44). Like fer-1, dysferlin is a large protein with an extensive, highly charged hydrophilic region and a single predicted membrane spanning region at the carboxy terminus (FIG. 3). There is a membrane retention sequence 3′ to the membrane spanning stretch, indicating that the protein may be preferentially targeted to either endoplasmic or sarcoplasmic reticulum, probably as a Type II protein (i.e. with the NH₂ end and most of the following protein located within the cytoplasm) (FIG. 1C). Several nuclear membrane targeting sequences are predicted within the cytoplasmic domain of the protein (http://psort.nibb.ac.jr/form.html). Immunocytochemical detection of dysferlin suggests that dysferlin is targeted to or anchored within the sarcoplasmic reticulum.

The cytoplasmic component of this protein contains four motifs homologous to C2 domains. C2 domains are intracellular protein modules composed of 80-130 amino acids (Rizo & Sudhof, 1998, J. Biol. Chem. 273:15897). Originally identified within a calcium-dependent isoform of protein kinase C (Nishizaka, 1988, Nature 334:661-65), C2 domains are present in numerous proteins. These domains often arise in approximately homologous pairs described as double C2 or DOC2 domains. One DOC2 protein, DOC2α, is brain specific and highly concentrated in synaptic vesicles (Orita et al., 1995, Biochem. Biophys. Res. Comm. 206:439-48), while another, DOC2β, is ubiquitously expressed (Sakaguchi et al., 1995, Biochem. Biophys. Res. Comm. 217:1053-61). Many C2 modules can fold to bind calcium, thereby initiating signaling events such as phospholipid binding. At distal nerve terminals, for example, the synaptic vesicle protein synaptotagmin has two C2 domains that, upon binding calcium, permit this protein to interact with syntaxin, triggering vesicle fusion with the distal membrane and neurotransmitter release (Sudhof & Rizo, 1996, Neuron 17:379-88).

The four dysferlin C2 domains are located at amino acid positions 32-82, 431-475, 1160-1241, and 1582-1660 (FIGS. 1C and 3). Indeed, it is almost exclusively through these regions that dysferlin has homology to any proteins other than fer-1. Each of these segments in dysferlin is considerably smaller than a typical C2 domain. Moreover, these segments are more widely separated in comparison with the paired C2 regions in synaptotagmin, DOC2α and β and related C2-positive proteins. For this reason, it is difficult to predict whether the four relatively short C2 domains in dysferlin function analogously to conventional C2 modules. That dysferlin might, by analogy with synaptotagmin, signal events such as membrane fusion is suggested by the fact that fer-1 deficient worms show defective membrane organelle fusion within spermatozoa (Ward et al., 1981, J. Cell Bio. 91:26-44).

The invention will be further described in the following examples, which do not limit the scope of the invention described in the claims.

### EXAMPLE 1

### Production of Dysferlin Protein

Standard methods can be used to synthesize either wild type or mutant dysferlin, or fragments of either. For example, a recombinant expression vector encoding dysferlin (or a fragment thereof: e.g., dysferlin minus its membrane-spanning region) operably linked to appropriate expression control sequences can be used to express dysferlin in a prokaryotic (e.g., E. coli) or eukaryotic host (e.g., insect cells, yeast cells, or mammalian cells). The protein is then purified by standard techniques. If desired, DNA encoding part or all of the dysferlin sequence can be joined in-frame to DNA encoding a different polypeptide, to produce a chimeric DNA that encodes a hybrid polypeptide. This can be used, for example, to add a tag that will simplify identification or purification of the expressed protein, or to render the dysferlin (or fragment thereof) more immunogenic.

The preferred means for making short peptide fragments of dysferlin is by chemical synthesis. These fragments, like dysferlin itself, can be used to generate antibodies, or as positive controls for antibody-based assays.

### EXAMPLE 2

### Production of Anti-Dysferlin Antibodies

Techniques for generating both monoclonal and polyclonal antibodies specific for a particular protein are well known. The antibodies can be raised against a short peptide epitope of dysferlin, an epitope linked to a known immunogen to enhance immunogenicity, a long fragment of dysferlin, or the intact protein. Such antibodies are useful for e.g., localizing dysferlin polypeptides in tissue sections or fractionated cell preparations and diagnosing dysferlin-related disorders.

An isolated dysferlin protein, or a portion or fragment thereof, can be used as an immunogen to generate antibodies that bind dysferlin using standard techniques for polyclonal and monoclonal antibody preparation. The dysferlin immunogen can also be a mutant dysferlin or a fragment of a mutant dysferlin. A full-length dysferlin protein can be used or, alternatively, antigenic peptide fragments of dysferlin can be used as immunogens. The antigenic peptide of dysferlin comprises at least 8 (preferably 10, 15, 20, or 30) amino acid residues of the amino acid sequence shown in SEQ ID NO: 2 and encompasses an epitope of such that an antibody raised against the peptide forms a specific immune complex with dysferlin. Preferred epitopes encompassed by the antigenic peptide are regions of dysferlin that are located on the surface of the protein, e.g., hydrophilic regions.

A dysferlin immunogen typically is used to prepare antibodies by immunizing a suitable subject (e.g., rabbit, goat, mouse or other mammal) with the immunogen. An appropriate immunogenic preparation can contain, for example, recombinantly expressed dysferlin protein or a chemically synthesized dysferlin polypeptide. The preparation can further include an adjuvant, such as Freund's complete or incomplete adjuvant, or similar immunostimulatory agent. Immunization of a suitable subject with an immunogenic dysferlin preparation induces a polyclonal anti-dysferlin antibody response.

Polyclonal anti-dysferlin antibodies (“dysferlin antibodies”) can be prepared as described above by immunizing a suitable subject with a dysferlin immunogen. The dysferlin antibody titer in the immunized subject can be monitored over time by standard techniques, such as with an enzyme linked immunosorbent assay (ELISA) using immobilized dysferlin. If desired, the antibody molecules directed against dysferlin can be isolated from the mammal (e.g., from the blood) and further purified by well-known techniques, such as protein A chromatography to obtain the IgG fraction. At an appropriate time after immunization, e.g., when the dysferlin antibody titers are highest, antibody-producing cells can be obtained from the subject and used to prepare monoclonal antibodies by standard techniques, such as the hybridoma technique originally described by Kohler and Milstein (1975) Nature 256:495-497, the human B cell hybridoma technique (Kozbor et al. (1983) Immunol. Today 4:72), the EBV-hybridoma technique (Cole et al. (1985), Monoclonal Antibodies and Cancer Therapy, Alan R. Liss, Inc., pp. 77-96) or trioma techniques. The technology for producing hybridomas is well known (see generally Current Protocols in Immunology (1994) Coligan et al. (eds.) John Wiley & Sons, Inc., New York, N.Y.). Briefly, an immortal cell line (typically a myeloma) is fused to lymphocytes (typically splenocytes) from a mammal immunized with a dysferlin immunogen as described above, and the culture supernatants of the resulting hybridoma cells are screened to identify a hybridoma producing a monoclonal antibody that binds dysferlin.

Any of the many well known protocols used for fusing lymphocytes and immortalized cell lines can be applied for the purpose of generating a monoclonal antibody against dysferlin (see, e.g., Current Protocols in Immunology, supra; Galfre et al. (1977) Nature 266:55052; R. H. Kenneth, in Monoclonal Antibodies: A New Dimension In Biological Analyses, Plenum Publishing Corp., New York, N.Y. (1980); and Lerner (1981) Yale J. Biol. Med., 54:387-402. Moreover, the one in the art will appreciate that there are many variations of such methods which also would be useful. Hybridoma cells producing a monoclonal antibody of the invention are detected by screening the hybridoma culture supernatants for antibodies that bind dysferlin, e.g., using a standard ELISA assay.

Alternative to preparing monoclonal antibody-secreting hybridomas, a monoclonal dysferlin antibody can be identified and isolated by screening a recombinant combinatorial immunoglobulin library (e.g., an antibody phage display library) with dysferlin to thereby isolate immunoglobulin library members that bind dysferlin. Kits for generating and screening phage display libraries are commercially available (e.g., the Pharmacia Recombinant Phage Antibody System, Catalog No. 27-9400-01; and the Stratagene SurfZAP™ Phage Display Kit, Catalog No. 240612). Additionally, examples of methods and reagents particularly amenable for use in generating and screening antibody display library can be found in, for example, U.S. Pat. No. 5,223,409; PCT Publication No. WO 92/18619; PCT Publication No. WO 91/17271; PCT Publication No. WO 92/20791; PCT Publication No. WO 92/15679; PCT Publication No. WO 93/01288; PCT Publication No. WO 92/01047; PCT Publication No. WO 92/09690; PCT Publication No. WO 90/02809; Fuchs et al. (1991) Bio/Technology 9:1370-1372; Hay et al. (1992) Hum. Antibod. Hybridomas 3:81-85; Huse et al. (1989) Science 246:1275-1281; Griffiths et al. (1993) EMBO J. 12:725-734.

### EXAMPLE 3

### Diagnosis

The discovery that defects in the dysferlin gene underlying the MM and LM(D2B phenotypes means that individuals can be tested for the disease gene before symptoms appear. This will permit genetic testing and counseling of those with a family history of the disease. Additionally, individuals, diagnosed with the genetic defect can be closely monitored for the appearance of symptoms, thereby permitting early intervention, including genetic therapy, as appropriate.

Diagnosis can be carried out on any suitable genomic DNA sample from the individual to be tested. Typically, a blood sample from an adult or child, or a sample of placental or umbilical cord cells of a newborn would be used; alternatively, one could utilize a fetal sample obtained by amniocentesis or chorionic villi sampling.

It is expected that standard genetic diagnostic methods can be used. For example, PCR can be utilized to identify the presence of a deletion, addition, or substitution of one or more nucleotides within any one of the exons of dysferlin. Following the PCR reaction, the PCR product can be analyzed by methods such as a heteroduplex detection technique based upon that of White et al. (1992, Genomics 12:301-06), or by techniques such as cleavage of RNA-DNA hybrids using RNase A (Myers et al., 1985, Science 230:1242-46), single-stranded conformation polymorphism (SSCP) analysis (Orita et al., 1989, Genomics 10:298-99), di-deoxy-fingerprinting (DDF) (Blaszyk et al., 1995, Biotechniques 18: 256-260) and denaturing gradient gel electrophoresis (DGGE; Myers et al., 1987, Methods Enzymol. 155:501-27). The PCR may be carried cut using a primer which adds a G+C rich sequence (termed a “GC-clamp”) to one end of the PCR product, thus improving the sensitivity of the subsequent DGGE procedure (Sheffield et al., 1989, Proc. Natl. Acad. Sci. USA 86:232-36). If the particular mutation present in the patient's family is known to have removed or added a restriction site, or to have significantly increased or decreased the length of a particular restriction fragment, a protocol based upon restriction fragment length polymorphism (RFLP) analysis (perhaps combined with PCR) may be appropriate.

The apparent genetic heterogeneity resulting in the MM/LGMD2B phenotypes means that the nature of the particular mutation carried by affected individuals in the patient's family may have to be ascertained prior to attempting genetic diagnosis of the patient. Alternatively, a battery of tests designed to identify any of several mutations known to result in MM/LGMD2B may be utilized to screen individuals without a defined familial genotype. The analysis can be carried out on any genomic DNA derived from the patient, typically from a blood simple.

Instead of basing the diagnosis on analysis of the genomic DNA of a patient, one could seek evidence of the mutation in the level or nature of the relevant expression products. Well-known techniques for analyzing expression include mRNA-based methods, such as Northern blots and in situ hybridization (using a nucleic acid probe derived from the relevant cDNA), and quantitative PCR (as described in St-Jacques et al., 1994, Endocrinology 134:2645-57). One could also employ polypeptide based methods, including the use of antibodies specific for the polypeptide of interest. These techniques permit quantitation of the amount of expression of a given gene in the tissue of interest, at least relative to positive and negative controls. One would expect an individual who is heterozygous for a genetic defect affecting the level of expression of dysferlin to show up to a 50% loss of expression of this gene in such a hybridization or antibody-based assay. An antibody specific for the carboxy terminal end would be likely to pick up (by failure to bind to) most or all frameshift and premature termination signal mutations, as well as deletions of the carboxy terminal sequence. Use of a battery of monoclonal antibodies specific for different epitopes of dysferlin would be useful for rapidly screening cells to detect those expressing mutant forms of dysferlin (i.e., cells which bind to some dysferlin-specific monoclonal antibodies, but not to others), or for quantifying the level of dysferlin on the surface of cells. One could also use a protein truncation assay (Heim et al., 1994, Nature Genetics 8:218-19) to screen for any genetic defect which results in the production of a truncated polypeptide instead of the wild type protein.

### EXAMPLE 4

### Therapeutic Treatment

A patient with MM/LGMD2B, or an individual genetically susceptible to contracting one or both of these diseases, can be treated by supplying dysferlin therapeutic agents of the present invention. Dysferlin therapeutic agents include a DNA or a subgenomic polynucleotide coding for a functional dysferlin protein. A DNA (e.g., a cDNA) is prepared which encodes the wild type form of the gene operably linked to expression control elements (e.g., promoter and enhancer) that induce expression in skeletal muscle cells or any other affected cells. The DNA may be incorporated into a vector appropriate for transforming the cells, such as a retrovirus, adenovirus, or adeno-associated virus. Alternatively, one of the many other known types of techniques for introducing DNA into cells in vivo may be used (e.g., liposomes). Particularly useful would be naked DNA techniques, since naked DNA is known to be readily taken up by skeletal muscle cells upon injection into muscle. Wildtype dysferlin protein can also be administered to an individual who either expresses mutant dysferlin protein or expresses an inadequate amount of dysferlin protein, e.g., a MM/LGMD2B patient.

Administration of the dysferlin therapeutic agents of the invention can include local or systemic administration, including injection, oral administration, particle gun, or catheterized administration, and topical administration. Various methods can be used to administer the therapeutic dysferlin composition directly to a specific site in the body. For example, a specific muscle can be located and the therapeutic dysferlin composition injected several times in several different locations within the body of the muscle.

The therapeutic dysferlin composition can be directly administered to the surface of the muscle, for example, by topical application of the composition. X-ray imaging can be used to assist in certain of the above delivery methods. Combination therapeutic agents, including a dysferlin protein or polypeptide or a subgenomic dysferlin polynucleotide and other therapeutic agents, can be administered simultaneously or sequentially.

Receptor-mediated targeted delivery of therapeutic compositions containing dysferlin subgenomic polynucleotides to specific tissues can also be used. Receptor-mediated DNA delivery techniques are described in, for example, Findeis et al. (1993), Trends in Biotechnol. 11, 202-05; Chiou et al. (1994), Gene Therapeutics: Methods and Applications of Direct Gene Transfer (J. A. Wolff, ed.); Wu & Wu (1988), J. Biol. Chem. 263, 621-24; Wu et al. (1994), J. Biol. Chem. 269, 542-46; Zenke et al. (1990), Proc. Natl. Acad. Sci. U.S.A. 87, 3655-59; Wu et al. (1991), J. Biol. Chem. 266, 338-42.

Alternatively, a dysferlin therapeutic composition can be introduced into human cells ex vivo, and the cells then implanted into the human. Cells can be removed from a variety of locations including, for example, from a selected muscle. The removed cells can then be contacted with the dysferlin therapeutic composition utilizing any of the above-described techniques, followed by the return of the cells to the human, preferably to or within the vicinity of a muscle. The above-described methods can additionally comprise the steps of depleting fibroblasts or other contaminating non-muscle cells subsequent to removing muscle cells from a human.

Both the dose of the dysferlin composition and the means of administration can be determined based on the specific qualities of the therapeutic composition, the condition, age, and weight of the patient, the progression of the disease, and other relevant factors. If the composition contains dysferlin protein or polypeptide, effective dosages of the composition are in the range of about 1 μg to about 100 mg/kg of patient body weight, e.g., about 50 μg to about 50 mg/kg of patient body weight, e.g., about 500 μg to about 5 mg/kg of patient body weight.

Therapeutic compositions containing dysferlin subgenomic polynucleotides can be administered in a range of about 0.1 μg to about 10 mg of DNA/dose for local administration in a gene therapy protocol. Concentration ranges of about 0.1 μg to about 10 mg, e.g., about 1 μg to about 1 mg, e.g., about 10 μg to about 100 μg of DNA can also be used during a gene therapy protocol. Factors such as method of action and efficacy of transformation and expression are considerations that will effect the dosage required for ultimate efficacy of the dysferlin subgenomic polynucleotides. Where greater expression is desired over a larger area of tissue, larger amounts of dysferlin subgenomic polynucleotides or the same amounts readministered in a successive protocol of administrations, or several administrations to different adjacent or close tissue portions of for example, a muscle site, may be required to effect a positive therapeutic outcome. In all cases, routine experimentation in clinical trials will determine specific ranges for optimal therapeutic effect.

### EXAMPLE 5

### Animal Model

A line of transgenic animals (e.g., mice, rats, guinea pigs, hamsters, rabbits, or other mammals) can be produced bearing a transgene encoding a defective form of dysferlin. Standard methods of generating such transgenic animals would be used, e.g., as described below.

Alternatively, standard methods of producing null (i.e., knockout) mice could be used to generate a mouse which bears one defective and one wild type allele encoding dysferlin. If desired, two such heterozygous mice could be crossed to produce offspring which are homozygous for the mutant allele. The homozygous mutant offspring would be expected to have a phenotype comparable to the human MM and/or LGMD2B phenotype, and so serve as models for the human disease.

For example, in one embodiment, dysferlin mutations are introduced into a dysferlin gene of a cell, e.g., a fertilized oocyte or an embryonic stem cell. Such cells can then be used to create non-human transgenic animals in which exogenous altered (e.g., mutated) dysferlin sequences have been introduced into their genome or homologously recombinant animals in which endogenous dysferlin nucleic acid sequences have been altered. Such animals are useful for studying the function and/or activity of dysferlin and for identifying and/or evaluating modulators of dysferlin function. As used herein, a “transgenic animal” is a non-human animal, preferably a mammal, more preferably a rodent such as a rat or mouse, in which one or more of the cells of the animal includes a transgene. Other examples of transgenic animals include non-human primates, sheep, dogs, cows, goats, chickens, amphibians, etc. A transgene is exogenous DNA which is integrated into the genome of a cell from which a transgenic animal develops and which remains in the genome of the mature animal, thereby directing the expression of an encoded gene product in one or more cell types or tissues of the transgenic animal. As used herein, an “homologously recombinant animal” is a non-human animal, preferably a mammal, more preferably a mouse, in which an endogenous dysferlin gene has been altered by homologous recombination between the endogenous gene and an exogenous DNA molecule introduced into a cell of the animal, e.g., an embryonic cell of the animal, prior to completed development of the animal.

A transgenic animal of the invention can be created by introducing a nucleic acid encoding a dysferlin mutation into the male pronuclei of a fertilized oocyte, e.g., by microinjection or retroviral infection, and allowing the oocyte to develop in a pseudopregnant female foster animal. A dysferlin cDNA sequence e.g., that of (SEQ ID NO: 1 or SEQ ID NO: 3) can be introduced as a transgene into the genome of a non-human animal. Alternatively, a nonhuman homologue of the human dysferlin gene can be isolated based on hybridization to the human dysferlin sequence (e.g., cDNA) and used as a transgene. Intronic sequences and polyadenylation signals can also be included in the transgene to increase the efficiency of expression of the transgene. Methods for generating transgenic animals via embryo manipulation and microinjection, particularly animals such as mice, have become conventional in the art and are described, for example, in U.S. Pat. Nos. 4,736,866 and 4,870,009, U.S. Pat. No. 4,873,191 and in Hogan, Manipulating the Mouse Embryo, (Cold Spring Harbor Laboratory Press, Cold Spring Harbor, N.Y., 1986). Similar methods are used for production of other transgenic animals. A transgenic founder animal can be identified based upon the presence of the mutant dysferlin transgene in its genome and/or expression of the mutant dysferlin mRNA in tissues or cells of the animals. A transgenic founder animal can then be used to breed additional animals carrying the transgene. Moreover, transgenic animals carrying a transgene encoding a mutant dysferlin can further be bred to other transgenic animals carrying other transgenes.

To create an homologously recombinant animal, a vector is prepared which contains at least a portion of a dysferlin gene into which a deletion, addition or substitution has been introduced to thereby alter a dysferlin gene. In a preferred embodiment, thus vector is designed such that, upon homologous recombination, the endogenous dysferlin gene is functionally disrupted (i.e., no longer encodes a functional protein; also referred to as a “knock out” vector). Alternatively, the vector can be designed such that, upon homologous recombination, the endogenous dysferlin gene is mutated or otherwise altered (e.g., contains one of the mutations described in Table 2). In the homologous recombination vector, the altered portion of the dysferlin sequence is flanked at its 5′ and 3′ ends by additional nucleic acid of the dysferlin gene to allow for homologous recombination to occur between the exogenous dysferlin nucleic acid sequence carried by the vector and an endogenous dysferlin gene in an embryonic stem cell. The additional flanking dysferlin nucleic acid is of sufficient length for successful homologous recombination with the endogenous gene. Typically, several kilobases of flanking DNA (both at the 5′ and 3′ ends) are included in the vector (see, e.g., Thomas and Capecchi (1987) Cell 51:503 for a description of homologous recombination vectors). The vector is introduced into an embryonic stem cell line (e.g., by electroporation) and cells in which the introduced dysferlin sequence has homologously recombined with the endogenous dysferlin gene are selected (see, e.g., Li et al. (1992) Cell 69:915). The selected cells are then injected into a blastocyst of an animal (e.g., a mouse) to form aggregation chimeras (see, e.g., Bradley in Teratocarcinomas and Embryonic Stem Cells: A Practical Approach, Robertson, ed. (IRL, Oxford, 1987) pp. 113-152). A chimeric embryo can then be implanted into a suitable pseudopregnant female foster animal and the embryo brought to term. Progeny harboring the homologously recombined DNA in their germ cells can be used to breed animals in which all cells of the animal contain the homologously recombined DNA by germline transmission of the transgene. Methods for constructing homologous recombination vectors and homologous recombinant animals are described further in Bradley (1991) Current Opinion in Bio/Technology 2:823-829 and in PCT Publication Nos. WO 90/11354, WO 91/01140, WO 92/0968, and WO 93/04169.

Other Embodiments

It is to be understood that while the invention has been described in conjunction with the detailed description thereof, the foregoing description is intended to illustrate and not limit the scope of the invention, which is defined by the scope of the appended claims. Other aspects, advantages, and modifications are within the scope of the following claims.

What is claimed is:

1. An isolated DNA of 20-25 nucleotides in length comprising a nucleotide sequence selected from the group consisting of nucleotides 911-930 of SEQ ID NO: 1, 929-948 of SEQ ID NO: 1, 1019-1038 of SEQ ID NO: 1, 1392-1411 of SEQ ID NO: 1, 1424-1443 of SEQ ID NO: 1, 1484-1503 of SEQ ID NO: 1, 1499-1518 of SEQ ID NO: 1, 1543-1565 of SEQ ID NO: 1, 1715-1734 of SEQ ID NO: 1, 174)-1759 of SEQ ID NO: 1, 2241-2260 of SEQ ID NO: 1, 2864-2883 of SEQ ID NO: 1, 2978-2997 of SEQ ID NO: 1, 3057-3076 of SEQ ID NO: 1, 3198-3217 of SEQ ID NO: 1, 3252-3271 of SEQ ID NO: 1, 4356-4375 of SEQ ID NO: 1, 4665-4684 of SEQ ID NO: 1, 5016-5034 of SEQ ID NO: 1, 5610-5629 of SEQ ID NO: 1, 5726-5735 of SEQ ID NO: 1, 6035-6054 of SEQ ID NO: 1, 6179-6198 of SEQ ID NO: 1, 6243-6263 of SEQ ID NO: 1, and 6529-6548 of SEQ ID NO: 1.

2. An isolated DNA comprising a nucleotide sequence selected from the group consisting of
nucleotides 911-930 of SEQ ID NO: 1, wherein nucleotide C at 920 is T;
929-948 of SEQ ID NO: 1, wherein nucleotide C at 938 is G;
1019-1038 of SEQ ID NO: 1, wherein nucleotide G at 1028 is T;
1392-1411 of SEQ ID NO: 1, wherein nucleotide T at 1401 is C;
1424-1443 of SEQ ID NO: 1, wherein nucleotide A at 1433 is C;
1499-1518 of SEQ ID NO: 1, wherein nucleotide G at 1508 is T;
1715-1734 of SEQ ID NO: 1, wherein nucleotide A at 1724 is G;
1740-1759 of SEQ ID NO: 1, wherein nucleotide T at 1749 is C;
2241-2260 of SEQ ID NO: 1, wherein nucleotide T at 2250 is C;
2864-2883 of SEQ ID NO: 1, wherein nucleotide A at 2873 is G;
2978-2997 of SEQ ID NO: 1, wherein nucleotide G at 2987 is A;
3057-3076 of SEQ ID NO: 1, wherein nucleotide G at 3066 is A;
3198-3217 of SEQ ID NO: 1, wherein nucleotide G at 3207 is T;
3252-3271 of SEQ ID NO: 1, wherein nucleotide G at 3261 is T;
4356-4375 of SEQ ID NO: 1, wherein nucleotide G at 4365 is T;
5015-5034 of SEQ ID NO: 1, wherein nucleotide A at 5024 is G,
5610-5629 of SEQ ID NO: 1, wherein nucleotide G at 5619 is A;
5726-5735 of SEQ ID NO: 1, wherein nucleotide G at 5735 is T;
6035-6054 of SEQ ID NO: 1, wherein nucleotide G at 6044 is T;
6179-6198 of SEQ ID NO: 1, wherein nucleotide G at 6188 is T; and
6243-6263 of SEQ ID NO: 1, wherein nucleotide T at 6253 is deleted.

3. An isolated DNA comprising a nucleotide sequence selected from the group consisting of GCAGGTGCGTGGGATGGACG (SEQ ID NO: 232) and CATATCCTCTTCATCCCTGC (SEQ ID NO: 233).

4. A pair of single stranded oligonucleotides selected from the group consisting of SEQ ID NOs: 234-283, wherein the oligonucleotides are not the same.

5. A method for identifying a patient, a fetus, or a pre-embryo at risk for a dysferlin-associated disorder comprising:
a. providing a biological sample from the patient, fetus, or pre-embryo, and
b. determining whether the sample contains a mutation in a dysferlin gene, wherein the mutation is selected from the group consisting of
nucleotide C at 920 of SEQ ID NO: 1 is T,
nucleotide C at 938 of SEQ ID NO: 1 is G,
nucleotide G at 1028 of SEQ ID NO: 1 is T.
nucleotide T at 1401 of SEQ ID NO: 1 is C,
nucleotide A at 1433 of SEQ ID NO: 1 is C,
nucleotide G at 1508 of SEQ ID NO: 1 is T,
nucleotide A at 1724 of SEQ ID NO: 1 is G,
nucleotide T at 1749 of SEQ ID NO: 1 is C,
nucleotide T at 2250 of SEQ ID NO: 1 is C,
nucleotide A at 2873 of SEQ ID NO: 1 is G,
nucleotide G at 2987 of SEQ ID NO: 1 is A,
nucleotide G at 3066 of SEQ ID NO: 1 is A,
nucleotide G at 3207 of SEQ ID NO: 1 is T,
nucleotide G at 3261 of SEQ ID NO: 1 is T,
nucleotide G at 4365 of SEQ ID NO: 1 is T,
nucleotide A at 5024 of SEQ ID NO: 1 is G,
nucleotide G at 5619 of SEQ ID NO: 1 is A,
nucleotide G at 5735 of SEQ ID NO: 1 is T,
nucleotide G at 6044 of SEQ ID NO: 1 is T,
nucleotide G at 6188 of SEQ ID NO: 1 is T,
nucleotide T at 6253 of SEQ ID NO: 1 is deleted,
an insertion of GTCCGTGGG at 1553 of SEQ ID NO: 1, and
an insertion of ATCCTCTTCATC at 6538 of SEQ ID NO: 1,
the presence of the mutation indicating that the patient, fetus, or pre-embryo is at risk for a dysferlin-related disorder.

6. The method of claim 5, wherein the biological sample contains genomic DNA, said method comprising:
(a) incubating the sample with a restriction enzyme; and
(b) detecting the presence or absence of a restriction enzyme site in the sample as an indication of the presence or absence of a particular mutation in the sample, wherein the restriction enzyme is selected from the group consisting of BanII, Bsp1286I, RsaI, HhaI, HaeIII, Bsp1286, NlaIV, NlaIII, BcgI, AvaII, BstEII, PleI, HaeI, AluI, ApoI, Tsp509I, SalI, HincII, TaqI, HinfI, TfiI, SfaNI, and FokI sites.

7. A transgenic non-human mammal having a transgene which encodes a mutated dysferlin gene, wherein the mutated dysferlin gene contains one or more mutations selected from the group consisting of
nucleotide C at 920 of SEQ ID NO: 1 is T,
nucleotide C at 938 of SEQ ID NO: 1 is G,
nucleotide G at 1028 of SEQ ID NO: 1 is T.
nucleotide T at 1401 of SEQ ID NO: 1 is C,
nucleotide A at 1433 of SEQ ID NO: 1 is C,
nucleotide G at 1508 of SEQ ID NO: 1 is T,
nucleotide A at 1724 of SEQ ID NO: 1 is G,
nucleotide T at 1749 of SEQ ID NO: 1 is C,
nucleotide T at 2250 of SEQ ID NO: 1 is C,
nucleotide A at 2873 of SEQ ID NO: 1 is G,
nucleotide G at 2987 of SEQ ID NO: 1 is A,
nucleotide G at 3066 of SEQ ID NO: 1 is A,
nucleotide G at 3207 of SEQ ID NO: 1 is T,
nucleotide G at 3261 of SEQ ID NO: 1 is T,
nucleotide G at 4365 of SEQ ID NO: 1 is T,
nucleotide A at 5024 of SEQ ID NO: 1 is G,
nucleotide G at 5619 of SEQ ID NO: 1 is A,
nucleotide G at 5735 of SEQ ID NO: 1 is T,
nucleotide G at 6044 of SEQ ID NO: 1 is T,
nucleotide G at 6188 of SEQ ID NO: 1 is T,
nucleotide T at 6253 of SEQ ID NO: 1 is deleted,
an insertion of GTGCGTGGG at 1553 of SEQ ID NO: 1, and
an insertion of ATCCTCTTCATC at 6538 of SEQ ID NO: 1.
